(19) United States
(12) Patent Application Publication
(10) Pub. No.: US 2020/0101286 A1
(43) Pub. Date: Apr. 2, 2020

(54) Devices And Methods For The Generation Of Alerts Due To Rising Levels Of Circulating Ketone Bodies In Physiological Fluids

(71) Applicant: Biolinq, Inc., San Diego, CA (US)

(72) Inventors: Joshua Windmiller, Del Mar, CA (US); Jared Rylan Tangney, Encinitas, CA (US); Thomas Arnold Peyser, Menlo Park, CA (US); John Sjolund, San Diego, CA (US)

(73) Assignee: Biolinq, Inc., San Diego, CA (US)

(21) Appl. No.: 16/701,784

(22) Filed: Dec. 3, 2019

Related U.S. Application Data
(63) Continuation-in-part of application No. 16/666,259, filed on Oct. 28, 2019.
Application No. 16/666,259 is a continuation of application No. 16/152,372, filed on Oct. 4, 2018, now Pat. No. 10,492,708.
Application No. 16/152,372 is a continuation of application No. 15/590,105, filed on May 9, 2017, now Pat. No. 10,092,207.
(60) Provisional application No. 62/777,053, filed on Dec. 7, 2018.
(60) Provisional application No. 62/336,724, filed on May 15, 2016.

(51) Int. Cl. A61N 1/30; A61B 5/05; A61B 5/145; A61M 5/172; A61B 5/1468; A61N 1/05

(52) U.S. Cl. CPC A61N 1/30; A61B 5/05; A61B 5/14546; A61B 2562/125; A61B 5/1468; A61N 1/05; Y02E 60/527; A61M 5/1723

(57) ABSTRACT

Ketoacidosis is a medical emergency that requires swift intervention to avert life-threatening sequel. A body-worn sensor (50) configured to measure the levels of a ketone compound circulating in a physiological fluid of a wearer and capable of generating an alert to the wearer if the level of the circulating ketone compound exceeds a pre-defined level or rate of change is disclosed herein. The sensor (50) preferably includes at least one of an electrochemical sensor, an optical sensor, a galvanic sensor, a voltammetric sensor, an amperometric sensor, a potentiometric sensor, an impedimetric sensor, a resistive sensor, a capacitive sensor, an ultrasonic sensor, a radio-frequency sensor, or a microwave sensor.

### CROSS REFERENCES TO RELATED APPLICATIONS

The Present Application claims priority to U.S. Provisional Patent Application No. 62/777,053, filed on Dec. 7, 2018, and the Present Application is a continuation-in-part application of U.S. patent application Ser. No. 16/666,259, filed on Oct. 28, 2019, which is a continuation application of U.S. patent Ser. No. 16/152,372, filed on Oct. 4, 2018, now U.S. Pat. No. 10,492,708 issued on Dec. 3, 2019, which is a continuation application of U.S. patent Ser. No. 15/590,105, filed on May 9, 2017, now U.S. Pat. No. 10/092,207, issued on Oct. 9, 2018, which claims priority to U.S. Provisional Patent Application No. 62/336,724, filed on May 15, 2016, now expired, each of which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The technology described herein involves devices and methods for generating actionable alerts to a user via measurement of circulating ketone body levels in physiological fluids of said user by means of a skin-worn analyte-selective sensor.

### Description of the Related Art

Ketone bodies, water-soluble molecules produced from the liver during excessive consumption of fatty acids, are a normal byproduct of the energy metabolism during gluconeogenesis, the production of glucose (the body's primary source of energy) from non-carbohydrate sources. In healthy individuals, circulating ketone levels are typically low, albeit during periods of low food intake (fasting), low carbohydrate consumption, starvation, prolonged intense exercise, or high alcohol intake, the levels of ketone bodies can rise as the liver's glycogen stores become depleted; this is typically referred to as ketosis and is relatively harmless, with new lines of research potentially suggesting benefits to a ketogenic lifestyle through diet and exercise. However, in individuals with type 1 diabetes mellitus, elevated ketone levels, which often arise due to a shortage of insulin, can invoke fatty acid synthesis, thereby causing a dramatic rise in circulating ketone body levels; this metabolic pathway can give rise to a potentially life-threatening condition known as diabetic ketoacidosis (DKA). DKA is often present in newly-diagnosed patients with diabetes and is a leading cause of mortality. In patients with previously diagnosed type 1 diabetes, DKA may also arise from acute insulin insufficiency either due to inadvertent omission of insulin doses, occluded insulin pump sets or prolonged periods of insulin suspension by automated insulin delivery systems. Concomitant illness may also be a contributing factor in the development of DKA. The outward manifestation of symptoms of DKA are not entirely apparent until the condition becomes severe; hospitalization becomes a necessity at this stage to avoid complications of DKA, which often includes excessive dehydration, tachycardia, hypotension, cerebral edema, and coma. DKA is the leading cause of hospitalization, morbidity, and death in children with type 1 diabetes mellitus.

Prior art solutions have been concerned with temporally-discrete or episodic measurement of ketone bodies in the urine or capillary blood with either colorimetric or electrochemical detection, respectively. Colorimetric techniques, while non-invasive, are largely qualitative and require that a test strip be compared with a color chart to determine a relative range of ketone bodies present in a sample, typically urine. Electrochemical techniques, on the other hand, are a bit more invasive, requiring a fingerstick capillary blood sample, albeit are quantitative when paired with a handheld meter. Newer methods of expired breath analysis of ketone bodies (typically acetone, which is highly volatile) have been enabled by hand-held instrumentation featuring embedded analyte-selective gas sensors or fuel cells. These systems are straightforward to operate and can provide a quantitative readout of ketone levels in expired breath, however, they are prone to numerous sources of error, including interference arising from dietary intake and oral hygiene. More globally, current methods of ketone determination require user action to isolate a sample and are purely episodic measurements at a single point in time. Accordingly, such systems fail to generate alerts since continuous, quasi-continuous, or online monitoring is not feasible using these platforms. In other words, the prior art requires that the user be aware that they are at risk for ketoacidosis and proactively undertake a measurement with the available methods. Under many circumstances, this approach is impractical and fails to provide users with a sufficiently timely measurement to eliminate the need for a doctor's visit, emergency room visit or a hospital admission. A passive and automated method of sensing ketones is preferred, especially when the symptoms associated with DKA do not become readily apparent.

Prior art products include: Colorimetric test strips (FIG. 3) for urine ketone determination: Ketostix® Reagent Strips for Urinalysis (Bayer Healthcare, Leverkusen, Del.); Electrochemical test strips for capillary blood ketone determination; Precision Xtra® Blood Glucose & Ketone Monitoring System (Abbott Laboratories, Lake Bluff, Ill.), which is intended to be used in conjunction with Precision Xtra Blood Ketone Test Strips (FIG. 4); nova Max Plus Blood Glucose Monitoring System (Nova Diabetes Care, Billerica, Mass.), which is intended to be used in conjunction with StatStrip® Glucose/Ketone Connectivity Meter (Nova Biomedical, Waltham, Mass.), which is intended to be used in conjunction with StatStrip Ketone Test Strips; StatStrip Glucose/Ketone Xpress2® Meter (Nova Biomedical, Waltham, Mass.), which is intended to be used in conjunction with StatStrip Ketone Test Strips; Keto-mojo® Ketone Meter (Keto-mojo, Napa, Calif.), which is intended to be used in conjunction with Keto-mojo Ketone Test Strips; FORA 6® Connect Blood Glucose and (3-Ketone Monitoring System (ForaCare Inc, Moorpark, Calif.), which is intended to be used in conjunction with FORA 6® Connect Blood (3-Ketone Test Strips; STAT-Site® M Beta-Hydroxybutyrate (BHB) Analyzer (EKF Diagnostics, Cardiff, UK), which is intended to be used in conjunction with STAT-Site® M (3-HB Test Strips; Expired breath analyzer for ketone determination; Ketonix® Breath Ketone Analyzer (Ketonix AB, Stockholm, SE) (FIG. 5); LEVLhome® or LEVLpro® device (Medamonitor LLC, Seattle, Wash.).

A prior art patent is Gerber et al., U.S. Pat. No. 9,958,409 for Systems And Methods For Multiple Analyte Analysis which discloses systems and methods for multiple analyte analysis. In one embodiment, a method includes determining concentrations of first and second analytes in a sample. The first and second analytes may be, for example, glucose and hydroxybutyrate. In this form, an indication related to the measured concentration of hydroxybutyrate is provided in response to determining that the concentration of hydroxybutyrate is above a predetermined value. In a further aspect of this form, a quantitative indication representative of the measured glucose concentration is automatically provided regardless of the value of the measured glucose concentration. In another embodiment, a system includes a meter configured to interact with a test element to assess first and second analytes in a sample. Further embodiments, forms, objects, features, advantages, aspects, and benefits are apparent from the description and drawings.

Another prior art patent reference is Deturk, U.S. Patent Publication 2015071994 for a Device For Determining Fat Expenditure From Levels Of Ketone Bodies That Have Passed Through The Skin And Methods For Determining The Same which discloses a sensing device having a first and second opening, a first semipermeable membrane having a first surface and a second surface, and a second semipermeable membrane having a third and fourth surface, a ketone body sensor, and a void. The first opening is juxtaposed to the first surface and the second opening is juxtaposed to the third surface. The space between the first and second openings is the void and wherein the ketone body sensor is positioned within the void. Gasses may permeate through the first opening and into the void to contact the sensor and exit the void through the second opening.

Another prior art patent reference is Crouther et al., U.S. Patent Publication 2015475094 for Methods for Analyte Monitoring Management And Analyte Measurement Data Management, and Articles of Manufacture Related Thereto, which discloses generally, methods of analyte monitoring management, and articles of manufacturing related thereto. The methods include receiving analyte measurement data and analyzing the analyte measurement data for health related parameters. Recommendations are determined for creating or modifying a treatment program based on the analysis, and provided within a user-interface that enables a user to create or modify the treatment program. Further, generally, methods of for managing analyte measurement data, and articles of manufacturing related thereto, are provided. The methods include receiving analyte measurement data that represent data collected over a time period, and analyzing the analyte measurement data for analyte episodes within that time period. Threshold based episodes and/or rate-of-change based episodes may be determined.

Another prior art patent reference is Ahmad, U.S. Patent Publication 2015136629 for a Ketone Measurement System and Related Method with Accuracy and Reporting Enhancement Features, which discloses a portable ketone measurement device measures ketone levels in breath samples or other bodily fluid samples of a user, and communicates the ketone measurements to an application that runs on a smartphone or other mobile device of the user. The application may communicate with, and report the measurements to, a remote server. One or more components of the system (e.g., the portable ketone measurement device, the mobile application, and/or the server) may, where appropriate, adjust the ketone measurements to compensate for ketone variations resulting from, e.g., the age of the user, a medical condition of the user, a missed medication event, or an interrupted sleep event. The application may, in some scenarios, withhold the display of a ketone measurement from the user until an authorization has been received from the server.

Another prior art patent reference is PCT Publication WO2018164886 for Systems, Devices, and Methods for Wellness and Nutrition Monitoring and Management using Analyte Data, which discloses systems, devices and methods are provided for the monitoring and management of an individual's wellness and nutrition using analyte data from an in vivo analyte sensor. Generally, a sensor control device is provided for wear on the body. The sensor control device can include an in vivo analyte sensor for measuring an analyte level in a bodily fluid, an accelerometer for measuring the physical activity level of the subject, as well as communications circuitry for wirelessly transmitting data to a reader device. Furthermore, disclosed herein are embodiments of various graphical user interfaces for displaying analyte metrics on a reader device, comparing the analyte response of various foods and/or meals, modifying daily nutrient recommendations based on analyte metrics and physical activity level measurements, and other features described herein. Additionally, the embodiments disclosed herein can be used to monitor various types of analytes.

### BRIEF SUMMARY OF THE INVENTION

The technology described herein involves devices and methods for generating actionable alerts to a user via continuous measurement of circulating ketone body levels in physiological fluids of said user by means of a skin-worn analyte-selective sensor.

One aspect of the present invention is a body-worn sensor configured to measure the levels of a ketone compound circulating in a physiological fluid of a wearer and capable of generating an alert to said wearer if the level of said circulating ketone compound exceeds a pre-defined level or rate of change.

Another aspect of the present nvention is a body-worn sensor configured to measure the levels of a ketone compound circulating in a physiological fluid of a wearer and capable of displaying to said wearer a continuous or quasi-continuous reading of said ketone compound circulating in said physiological fluid.

Yet another aspect of the present invention is a method of generating an alert to a wearer of a body-worn sensor, said alert indicative of a metabolic state of an elevated ketone compound.

Yet another aspect of the present invention is a method for determining the rising levels of circulating ketone bodies in physiological fluids. The method includes measuring a concentration of a ketone compound circulating in a physiological fluid of a wearer of a body-worn sensor device comprising one of an electrochemical sensor, an optical sensor, a galvanic sensor, a voltammetric sensor, n amperometric sensor, a potentiometric sensor, an impedimetric sensor, a resistive sensor, a capacitive sensor, an ultrasonic sensor, a radio-frequency sensor, and a microwave sensor. The method also includes storing the measurement in a memory of the body-worn device. The method also includes determining if the concentration level exceeds a pre-defined level, threshold, or rate of change from a previous measurement. The method also includes generating an alert if the concentration level exceeds a pre-defined level, threshold, or rate of change from a previous measurement.

The sensor preferably includes at least one of an electrochemical sensor, an optical sensor, a galvanic sensor, a voltammetric sensor, an amperometric sensor, a potentiometric sensor, an impedimetric sensor, a resistive sensor, a capacitive sensor, an ultrasonic sensor, a radio-frequency sensor, and a microwave sensor.

Yet another aspect of the present invention includes the incorporation of a biorecognition element, such as an enzyme, into the sensor in order to convert the presence of a ketone compound to a physically-quantifiable signal.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is an illustration of an exemplary body-worn sensor configured to measure the levels of a ketone compound circulating in a physiological fluid of a wearer; a wearer's smartphone, which is configured to receive wireless readout from said body-worn sensor, displays an alert to said wearer.

FIG. 2 is a diagrammatic representation of the fatty acid synthesis metabolic pathway with the three ketone body products delineated-acetone, acetoacetate, and D-β-hydroxybutyrate.

FIG. 3 is an illustration of a prior art detection of ketone bodies in the urine by means of disposable colormetric test strips.

FIG. 4 is an illustration of a prior art quantification of ketone bodies in fingerstick capillary blood samples by means of disposable electrochemical test strips.

FIG. 5 is an illustration of a prior art quantification of ketone bodies in expired breath by means of a handheld spirometer.

FIG. 6 is a block diagram illustrating the major constituents involved in the functionalization of a skin-penetrating electrochemical sensor to facilitate chemical or biochemical quantification of various analytes in physiological fluids.

FIG. 7 is a process flow diagram illustrating the major constituents involved in the generation of an alert to the wearer of a body-worn sensor based on the reading of one or more ketone compounds circulating in a physiological fluid.

FIG. 8 illustrates electronic circuitry contained in prototype wearable device enclosure designed to interface directly with a microneedle-based biosensor device.

FIG. 9 illustrates another view of the electronic circuitry contained in prototype wearable device enclosure designed to interface directly with a microneedle-based biosensor device.

FIG. 10 illustrates electronic circuitry contained in sealed housing with access to the microneedle device provided via gold-plated pressure connectors located on the viewable surface of the housing.

FIG. 11 illustrates a skin-penetrating hollow microneedle array comprising a plurality of protrusions having vertical extent of approximately 1000 μm, with each element of the microneedle array functionalized to impart selective biosensing ability.

FIG. 12A illustrates a hollow, unfunctionalized microneedle array.

FIG. 12B illustrates a hollow “filled”, functionalized microneedle array with selective biosensing ability.

FIG. 13 illustrates an exploded view rendering of complete microneedle biosensing system illustrating all functional components, including the microneedle biosensor and printed circuit board containing the electronic circuitry required to transduce biochemical signals to digital data that can be wirelessly transmitted to an external device via an embedded wireless transceiver.

FIG. 13A is an isolated enlarged view of the microneedle biosensor component of FIG. 13.

FIG. 14 illustrates another view of the wearable microneedle biosensing system containing the electronic backbone (protrusion) and adhesive patch, wherein the microneedle is located on the posterior surface of the adhesive patch (not shown).

FIG. 15 illustrates a posterior surface view of the electronics components housing constituent of the microneedle-based biosensing system and the skin-worn adhesive patch containing the microneedle array.

FIG. 16 illustrates a detailed block/process flow diagram illustrating the major functional components of the microneedle-based biosensing system and supporting electronic systems.

FIG. 17 is a circuit diagram of a standalone potentiostat integrated circuit.

FIG. 18 is a circuit diagram of a multi-component potentiostat.

FIG. 19 is a block diagram of a difference amplifier.

FIG. 20 is a signal flow diagram of the present invention.

FIG. 21 is a circuit diagram of an integrated analog front end and sensor interface.

FIG. 22 is a circuit diagram of mirrored difference amplifiers and filtering.

FIG. 23 is a circuit diagram of fixed mirrored instrumentation amplifiers.

FIG. 24 is a circuit diagram of digital potentiometer-adjustable mirrored instrumentation amplifiers.

FIG. 25 is an illustration of a handheld analyzer in a large form factor.

FIG. 26 is an illustration of a handheld analyzer in a small form factor.

FIG. 27 is a block diagram of a sample algorithm.

FIG. 28 is an illustration of a handheld analyzer in a small form factor.

### DETAILED DESCRIPTION OF THE INVENTION

In healthy individuals, circulating ketone levels are typically well below 0.5 milli-mol (“mM”). Slightly elevated ketone levels (i.e. between 0.5 and 1 mM) are typically a sign of ketosis, usually as a consequence of fasting or low-carbohydrate diets as the liver scavenges its fat reserves for energy. Healthy individuals are very rarely at risk for ketoacidosis (>1 mM, thereby causing acidification of the blood due to highly elevated levels of ketone bodies). The absence of insulin, which otherwise enables glucose to enter the cells to provide them with energy, causes the body to scavenge energy from free fatty acids in the liver, giving rise to excessive generation of ketone bodies and subsequent acidification of the blood, thereby disrupting acid/base homeostasis. Diabetic ketoacidosis (DKA) is a life-threatening metabolic complication of diabetes with a mortality rate of 2-10%. DKA usually manifested through extended periods of hyperglycemia and overall poor glycemic management, including insufficient administration of insulin, insufficient carbohydrate intake, and reactions to administered insulin. The risk of diabetic ketoacidosis is increased by concomitant illness. Gastroenteritis, for example, is a common precursor of DKA in patients with insulin-dependent diabetes for multiple reasons including the mistaken belief by some patients that if they are ill and unable to eat, they should reduce or eliminate their insulin intake. It is widely understood in clinical practice that early detection of increased blood ketone levels can help to avert DKA by enabling the patient to take numerous steps at home such as increased insulin delivery, increased hydration and other measures to prevent the development of acute illness and the need for emergency medical services and/or hospitalization.

The current solution provides for a body-worn sensor device and method to selectively quantify, in an automated and continuous fashion, a ketone body analyte in a physiological fluid of a wearer. Should the ketone body exceed a pre-defined threshold or rate of change, an alert is generated such that an action can be elicited in a timely fashion, thereby averting a potentially life-threatening incidence of ketoacidosis.

Ketoacidosis, most often a result of undiagnosed type 1 diabetes mellitus (T1D) or insulin insufficiency in previously-diagnostic T1D, is a medical emergency that requires swift intervention to avert life-threatening sequela. Physical presentation of signs of diabetic ketoacidosis (DKA) can be difficult to identify in the acute phase and often only materialize once dangerous levels of circulating ketone bodies have been attained. Indeed, only when manifestation of symptoms become readily apparent are individuals with T1D trained to test for ketone bodies with available episodic methods; it is often too late to avert hospitalization in such scenarios. In most cases, corrective action cannot be easily self-administered and a visit to a hospital emergency department is often necessary. As is evident, early identification of a potential episode of DKA in the acute phase (and subsequent swift corrective action) can mitigate the need for hospitalization or a visit to a local urgent care/emergency department. A promising class of drugs (sodium-glucose lowering transporter-2 (SGLT2) inhibitors) offers substantial potential to reduce blood sugar by instigating the kidneys to excrete circulating sugar through the urine, thereby helping individuals with T1D to achieve normal blood sugar levels (euglycemia, 70-180 mg/dL plasma glucose). Although this may seem beneficial for those with T1D, it has been observed that these drugs increase the risk for euglycemic diabetic ketoacidosis (DKA) in the absence of sustained hyperglycemia. Under these circumstances, individuals taking these medications may be unaware of dangerously elevated circulating ketone levels because their blood sugar appears to be well under control. Current methods for ketone analysis, either assessed via blood, urine, or breath sampling, do not enable continuous and passive assessment of circulating ketone bodies in these physiological compartments. Rather, the user must take proactive measures to test for ketones by means of actively sampling a physiological fluid or vapor. In this case, the detection of ketones are sporadic (rather than continuous) and that the presentation of an alert can only occur following an action by the user. In such a scenario, sampling during periods of sleep or activity is not feasible nor is it likely that the user will be able to identify an excursion in their ketone levels in the acute phase (i.e. shortly following a rise in circulating ketone bodies) owing to the often delayed symptoms of DKA. Moreover, alerts generated by a rate-of-change, derivative value, or trend analysis require a memory function to be implemented, whereby a knowledge of at least one past value must be made available for determination. In many cases, a past value might not be readily available as a prior assay may have occurred days, weeks, or months prior and thereby beyond the extent of an individual's recollection. The disclosed capability provides for a device and method for tendering alerts, in an automated and continuous fashion, to a wearer of a body-worn sensor should circulating ketone levels surpass a pre-defined threshold or rate of change. Through continuous online quantification of circulating ketone bodies in a physiological fluid, a body-worn sensor can identify potentially hazardous excursions in ketone levels, thereby potentially alerting the wearer to take a course of action that can swiftly bring ketone levels under control and avert DKA; such interventions are likely to improve outcomes and reduce morbidity and mortality of DKA. It is expected that this new, innovative, and clinically-useful capability will help improve the current standard of care in the diabetes domain, hence alleviating burdens on the patient, healthcare provider, and reimbursement infrastructure.

The device disclosed herein addresses the above challenges by the automated generation of alerts pertaining to an elevation of absolute ketone levels beyond a threshold or a ketone level that is increasing beyond a specified rate-of-change. This involves the application of an analyte-selective sensor on the body of a wearer, which is able to sample a physiological fluid compartment (i.e. blood, serum, plasma, interstitial fluid, dermal interstitial fluid, extracellular fluid, intracellular fluid, cerebrospinal fluid) for the presence of one or more circulating ketone bodies (acetoacetate, acetone, D-β-hydroxybutyrate). The sensor acquires the sample subcutaneously, percutaneously, transdermally, intradermally, or on the skin surface and employs an electrical or optical stimulus to encourage an electrical, photonic, or chemical change to occur; a voltage, current, charge, resistance, or impedance property is subsequently measured to infer the concentration of a singular ketone body or plurality of ketone bodies circulating in a physiological fluid compartment. A programmable memory element contained in the body-worn sensor contains a pre-programmed threshold value, here referring to an absolute value of a singular ketone body or plurality of ketone bodies. Furthermore, said element also retains past readings in order to provide a comparative assessment for slope, rate-of-change, derivative, or trending determinations. Should the value measured by said body-worn sensor exceed a pre-defined value or threshold, an alert is tendered to the wearer (and, optionally, their support network—family, healthcare provider, friends, etc). The said alert can take multiple forms—visual notification, audible notification, haptic notification, and a textual notification on the wearer's smartphone, smartwatch, wearable device, tablet, eyewear, earbud, or directly on the wearer's body-worn device. Trend/pattern analysis can be leveraged to predict future ketone excursions and machine learning can, likewise, be employed to identify scenarios that place the wearer of the body-worn device at risk for DKA. The said ketone alert-generation capability can also be paired with another sensing modality such as, for example, a continuous glucose monitor. The disclosed method facilitates the presentation of alerts to the wearer of a body-worn sensor device should the absolute level or rate-of-change of a singular ketone body (acetoacetate, acetone, D-β-hydroxybutyrate) or plurality of ketone bodies exceed a pre-defined threshold. Methods include the detection of said ketone body(ies) in a physiological fluid compartment of the wearer (i.e. blood, serum, plasma, interstitial fluid, deimal interstitial fluid, extracellular fluid, intracellular fluid, cerebrospinal fluid). The method can involve detection by means of a subcutaneous, percutaneous, transdermal, intradermal, or skin surface body-worn sensor, which is configured to employ an electrical or optical stimulus to encourage an electrical, photonic, or chemical change to occur; a voltage, current, charge, resistance, or impedance property is subsequently measured to infer the concentration of a singular ketone body or plurality of ketone bodies circulating in a physiological fluid compartment. A programmable memory element contained in said body-worn sensor contains a pre-programmed threshold value, here referring to an absolute value of a singular ketone body or plurality of ketone bodies. Furthermore, said element also retains past readings in order to provide a comparative assessment for slope, rate-of-change, derivative, or trending determinations. Should the value measured by said body-worn sensor exceed a pre-defined value or threshold, an alert is tendered.

Commercial products may be utilized in practicing the invention as pertaining to presenting the wearer of a body-worn sensor with an alert. Such commercial products include, but are not limited to, a smartphone (i.e. Apple iPhone, Samsung Galaxy phone), a smartwatch (i.e. Apple Watch, Fitbit versa), a wearable device (i.e. Fitbit Blaze, Garmin Forerunner), a tablet (i.e. Apple iPad, Samsung Galaxy Tab), an eyewear (i.e. Google Glass, Oculus Rift), an earbud (i.e. Apple Earpods, Bose SoundSport Free), a laptop (i.e. Apple MacBook, Dell XPS), a computer (i.e. Apple iMac, Lenovo ThinkCentre), or body-worn device (i.e. iRhythm Zio).

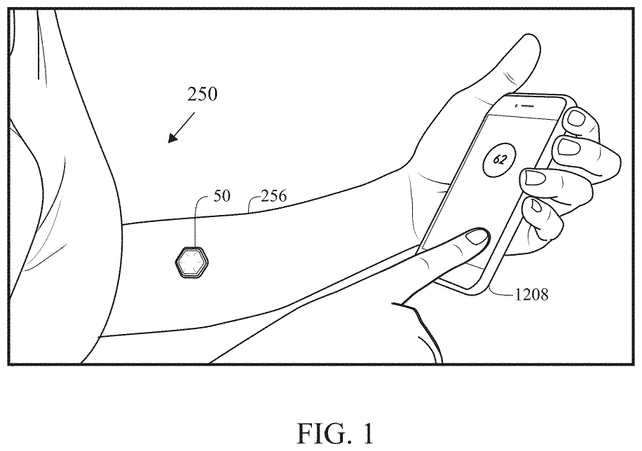

FIG. 1 is an illustration of an exemplary body-worn sensor 50 on an arm 256 of a wearer 250, and is configured to measure the levels of a ketone compound circulating in a physiological fluid of a wearer; a wearer's smartphone 1208, which is configured to receive wireless readout from the body-worn sensor 50, displays an alert to the wearer 250. The body-worn sensor 50 is preferably a subcutaneous, percutaneous, transdermal, intradermal, or skin surface sensor in which an electrical or optical stimulus is applied to encourage a redox reaction and in which a voltage, current, charge, resistance, or impedance property is measured to infer the concentration of a particular ketone body (acetoacetate, acetone, D-β-hydroxybutyrate) present in the physiological fluid compartment in which the sensor is located. The body-worn sensor 50 is preferably configured to contain an embedded memory for archiving past measurements. The body-worn sensor 50 is preferably configured to contain a wireless radio, transmitter, or transceiver to relay measurements to a paired wirelessly-enabled device.

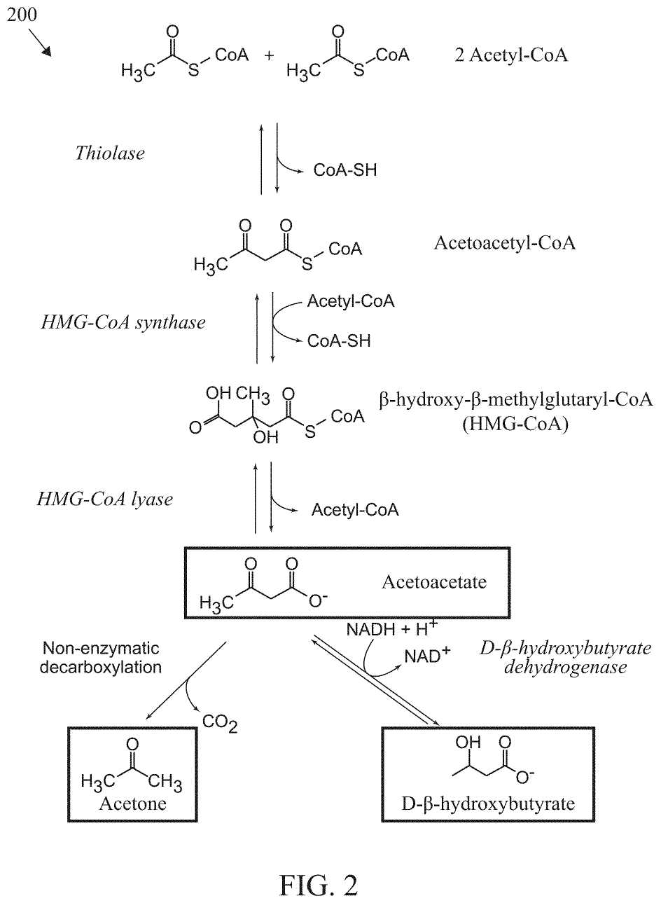

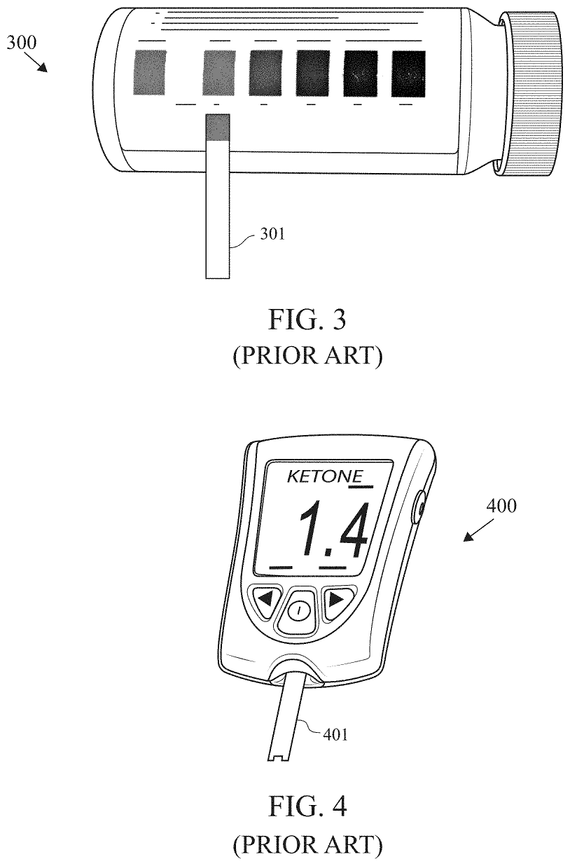

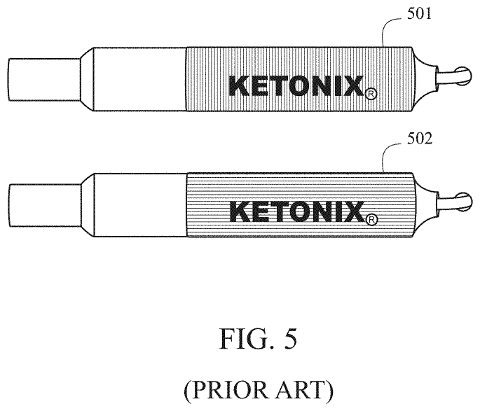

FIG. 2 is a diagrammatic representation of the fatty acid synthesis metabolic pathway with the three ketone body products delineated-acetone, acetoacetate, and D-β-hydroxybutyrate. FIG. 3 is an illustration of a prior art detection of ketone bodies in the urine by means 300 of disposable colormetric test strips 301. FIG. 4 is an illustration of a prior art quantification of ketone bodies in fingerstick capillary blood samples by means 400 of disposable electrochemical test strips 401. FIG. 5 is an illustration of a prior art quantification of ketone bodies in expired breath by means of a handheld spirometer 501 and 502.

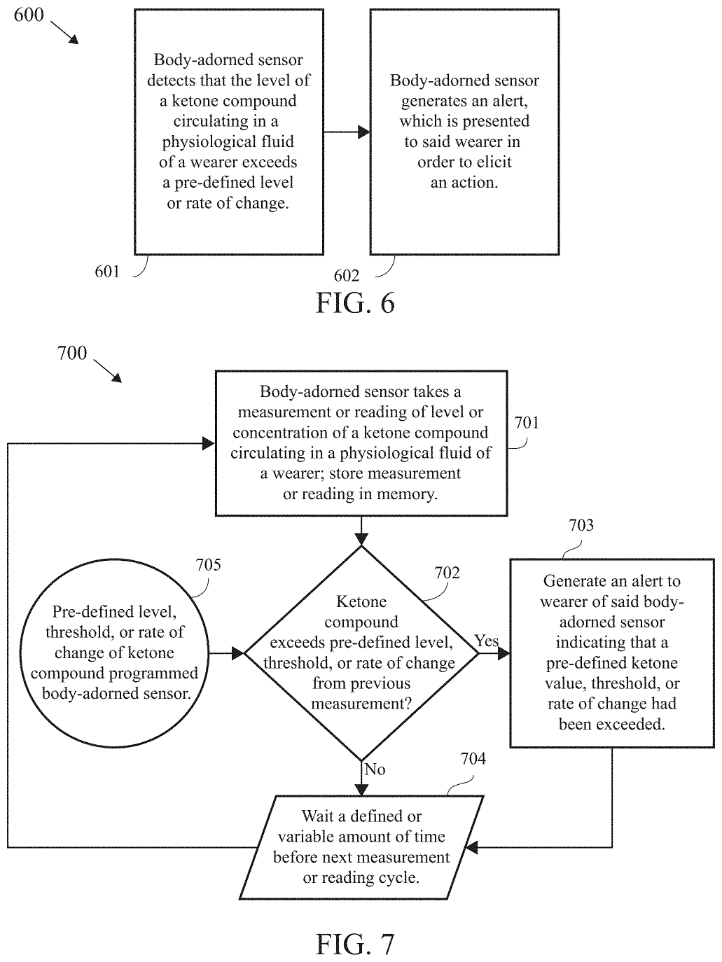

FIG. 6 is a block diagram illustrating the major constituents involved in the functionalization of a skin-penetrating electrochemical sensor to facilitate chemical or biochemical quantification of various analytes in physiological fluids. At block 601, a body-worn sensor determines if a ketone compound exceeds a pre-defined level, threshold, or rate of change from previous measurement. At block 602, the body-worn sensor generates an alert to the wearer of the body-worn sensor indicating that a pre-defined ketone value, threshold, or rate of change has been exceeded.

As shown in FIG. 7, a method for determining the rising levels of circulating ketones bodies in a physiological fluid includes at block 701 a body-worn sensor taking a measurement or reading of a level or a concentration of a ketone compound circulating in a physiological fluid of a wearer, and then storing the measurement or the reading in a memory of the body-worn sensor. Said sensor transduces the concentration of a circulating ketone compound in a physiological fluid of a wearer to a quantitative or qualitative value. Prior concentrations may be stored in a memory element to determine a rate of change, a derivative value, or a slope.

At block 702, the body-worn sensor determines if ketone compound exceeds pre-defined level, threshold, or rate of change from previous measurement. A comparative assessment is made between said measurement or reading and a pre-defined level, threshold, or rate of change from previous measurement inputted from block 705.

At block 703, if the action at block 702 is true, the body-worn sensor generates an alert to the wearer of said body-worn sensor indicating that a pre-defined ketone value, threshold, or rate of change has been exceeded. If the comparative assessment yields a TRUTH, an alert is generated to the wearer of the said body-worn sensor device indicative of a status of elevated ketone levels or increased ketone rate of change.

At block 704, if the action at block 702 is false, the body-worn sensor waits a defined or variable amount of time (based on absolute ketone level or rate of change of said ketone level) before a next measurement or a reading cycle. The body-worn sensor device operates in perpetuity to identify and subsequently alert the wearer if a ketone compound circulating in the physiological fluid of said wearer exceeds a pre-defined level, threshold, or rate of change from previous measurement.

An input is a ketone compound circulating in a physiological fluid of a wearer. A ketone compound can include at least one of acetone, acetone, acetoacetic acid, and β-hydroxybutyric acid. A physiological fluid can include blood, serum, plasma, interstitial fluid, dermal interstitial fluid, extracellular fluid, intracellular fluid, and cerebrospinal fluid.

A pre-defined threshold value, a level, a rate of change, a derivative value, a slope value,or a t d A quantified reference value in which the current measurement of a circulating ketone compound can be assessed against. If the value of the current measurement exceeds this reference value, then an alert will be generated.

An output is an alert. An alert preferably comprise at least one of a visual notification, audible notification, haptic notification, and a textual notification. The alert preferably indicates that at least one of a pre-defined threshold value, a level, a rate of change, a derivative value, or a slope value has been exceeded. The alert is preferably presented to the wearer by means of, but not limited to, a smartphone, a smartwatch, a wearable device, a tablet, an eyewear, an earbud, a laptop, a computer, or body-worn device. The alert is preferably tendered to elicit an action or be fore purely informational purposes only. The alert can also be conveyed to an individual or group of individuals in addition to the wearer by means of a communications network or Internet connection.

A microneedle-based biosensor is preferably implemented as a physical transducer/electrode to facilitate the transdermal analysis of pertinent biochemical analytes from the viable physiological medium (interstitial fluid, blood) occupying the layers of the epidermis and dermis. The electrochemical analog front end performs one (or more) of a number of electroanalytical techniques, such as voltammetry, amperometry, potentiometry, conductimetry, coulometry, impedimetry, and polarography, to facilitate the control and readout of the electrochemical reaction occurring at the microneedle-based biosensor. Optionally, the electrical signal generated at the output of the electrochemical analog front end is directed to an amplification circuit to increase the signal strength to line levels. Following this, the output is, optionally, directed to a low- or band-pass filter to extract the signal of interest and remove undesired noise. As an additional optional step, the signal subsequently undergoes analog-to-digital conversion to convert the analog signal to a digital bitstream. Finally, the signal is routed to a wireless transmitter or transceiver (Bluetooth, WiFi, RFID/NFC, Zigbee, Ant+) for transmission of the signal (corresponding to the level of the biochemical analyte) to a mobile or wearable device for further information processing, interpretation, display, archiving, and trending.

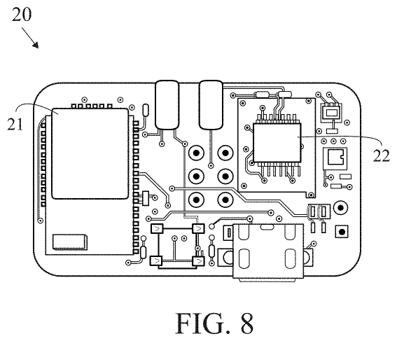

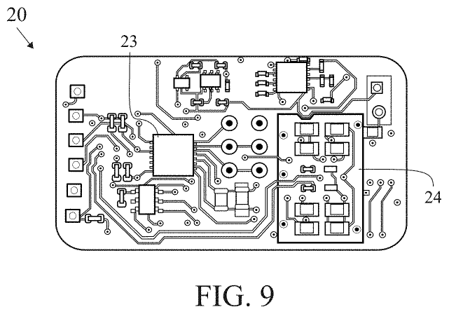

FIG. 8 illustrates the electronic circuitry contained in a wearable device enclosure 20 designed to interface directly with a microneedle-based biosensor device. The electronic circuitry of the device comprises a wireless transceiver (preferably BLUETOOTH LOW ENERGY) and a microcontroller with an integrated analog-to digital converter 21, and a high amplification circuit 22. FIG. 9 illustrates another view of the electronic circuitry contained in prototype wearable device enclosure 20 designed to interface directly with a microneedle-based biosensor device. The electronic circuitry comprises a high-sensitivity electrochemical analog front end 23 and a filtering circuit 24.

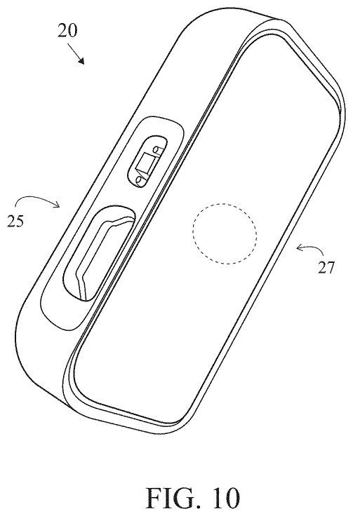

FIG. 10 illustrates the electronic circuitry contained in the wearable device enclosure 20 with access to the microneedle device provided via gold-plated pressure connectors 27 located on the viewable surface of the wearable device enclosure 20. A connection port 25 is also shown.

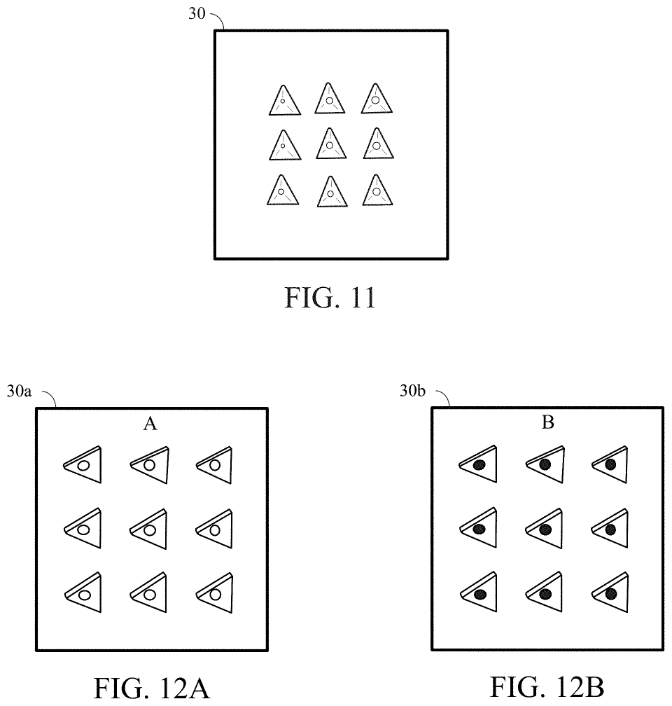

FIG. 11 illustrates a skin-penetrating hollow microneedle array 30 comprising a plurality of protrusions having vertical extent of approximately 1000 with each element of the microneedle array functionalized to impart selective biosensing ability. FIG. 12A illustrates a hollow, unfunctionalized microneedle array 30a. FIG. 12B illustrates a hollow “filled”, functionalized microneedle array 30b with selective biosensing ability.

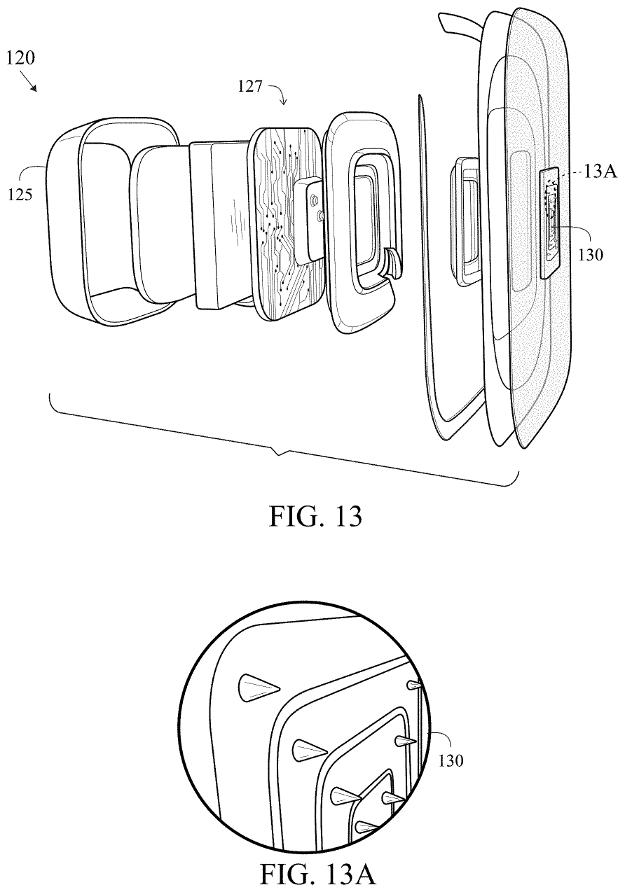

FIGS. 13 and 13A illustrate an exploded view rendering of complete microneedle biosensing system 120 illustrating the functional components, including a housing member 125, a microneedle biosensor 130 and a printed circuit board 127 containing the electronic circuitry required to transduce biochemical signals to digital data that are wirelessly transmitted to an external device via the embedded wireless transceiver.

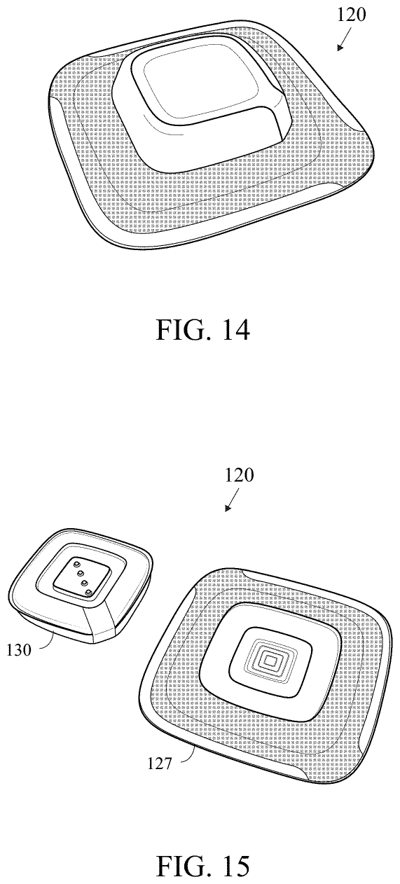

FIG. 14 illustrates a top perspective view of the wearable microneedle biosensing system 120 containing the electronic backbone (protrusion) and adhesive patch. The microneedle is located on the posterior surface of the adhesive patch (not shown).

FIG. 15 illustrates a posterior surface view of the electronics components housing constituent 130 of the microneedle-based biosensing system 120 and the skin-worn adhesive patch containing the microneedle array 127.

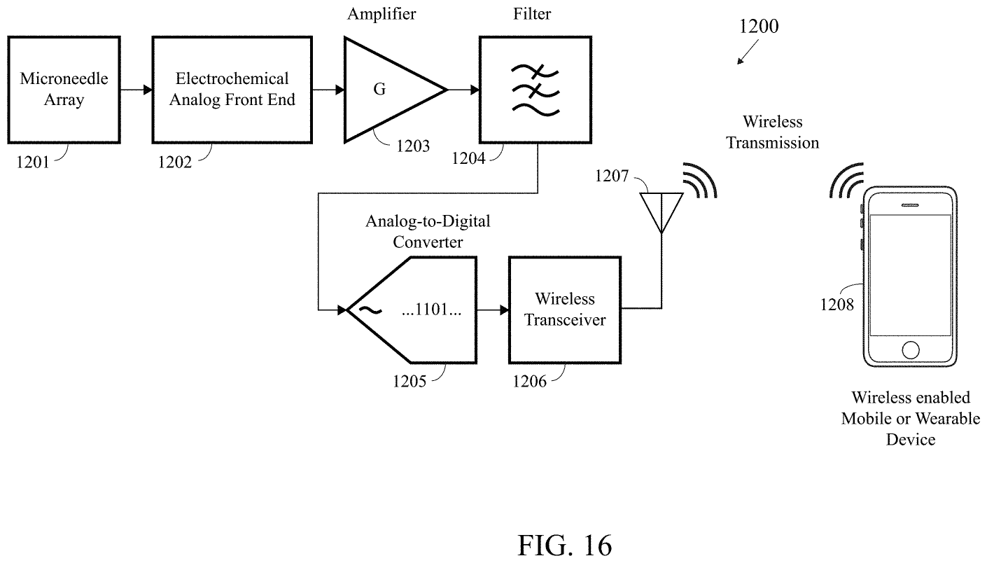

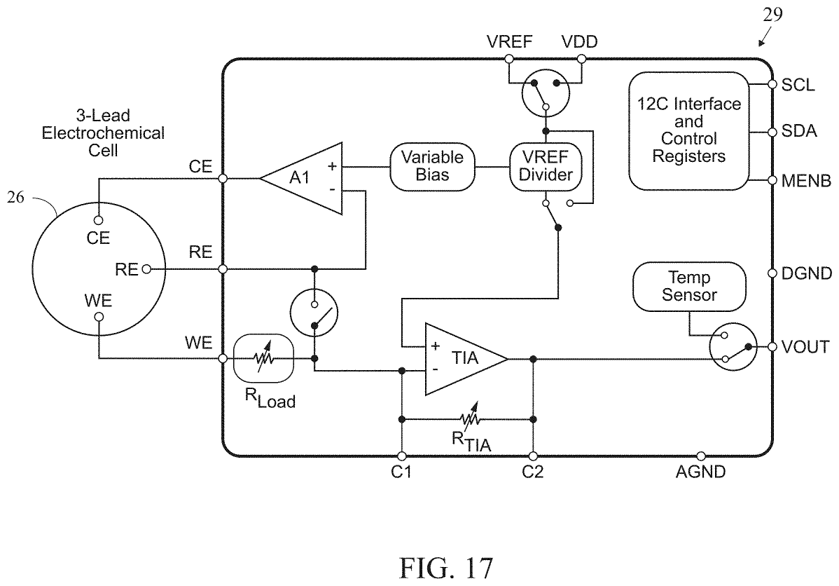

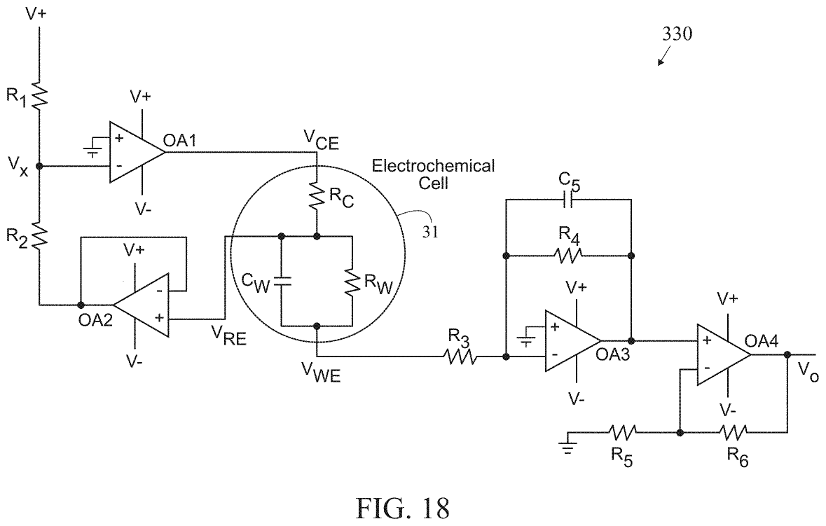

FIG. 16 illustrates a detailed block/process flow diagram 1200 illustrating the major functional components of the microneedle-based biosensing system and supporting electronic systems. At block 1201 is the microneedle array utilized to obtain transdermal biochemical analytes from a viable physiological medium (interstitial fluid, blood) occupying the layers of the epidermis and dermis of a user of the microneedle-based biosensing system. At block 1202, the electrochemical analog front end performs one (or more) of a number of electroanalytical techniques, such as voltammetry, amperometry, potentiometry, conductimetry, impedimetry, and polarography, to facilitate the control and readout of the electrochemical reaction occurring at the microneedle-based biosensing system. At block 1203, the electrical signal generated at the output of the electrochemical analog front end is directed to an amplification circuit to increase the signal strength to line levels. At block 1204, the output from the amplification circuit is directed to a low- or band-pass filter to extract a signal of interest and remove any undesired noise. At block 1205, the signal subsequently undergoes analog-to-digital conversion at an ADC to convert the analog signal to a digital bitstream. At block 1206, the signal is routed to a wireless transmitter or transceiver (BLUETOOTH, WiFi, RFID/NFC, Zigbee, Ant+) 1207 for transmission of the signal (corresponding to the level of the biochemical analyte) to a mobile communication device 1208 for further information processing, interpretation, display, archiving, and trending.

The electrochemical analog front end preferably includes: a Texas Instruments LMP91000 Sensor AFE System, configurable AFE potentiostat for low-power chemical sensing applications; a Texas Instruments LMP91200 configurable AFE for low-power chemical sensing applications; or an Analog Devices ADuCM350 16-Bit Precision, low power meter on a chip with Cortex-M3 and connectivity. The wireless transceiver is preferably is a BLUEGIGA BLE-113A BLUETOOTH Smart Module, or a Texas Instruments CC2540 SimpleLink BLUETOOTH Smart Wireless MCU with USB. The accompanying mobile device is preferably an ANDROID™-or iOS™-based smartphone, Samsung GALAXY GEAR, or an APPLE WATCH™.

The microneedle array electrochemical biosensor transduces biochemical signals from the interstitial fluid into useful electrical signals.

The electrochemical analog front end preferably performs at least one or more of the following: applies a fixed potential or time-varying potential to the microneedle array to induce an electrochemical reaction, thereby giving rise to a flow of current; applies a fixed current or time-varying current to the microneedle array to induce an electrochemical reaction, thereby giving rise to an electrical potential; measures a time-varying open-circuit potential generated by an electrochemical reaction or ionic gradient; measures a frequency-dependent impedance generated by an electrochemical or bio-affinity reaction at the microneedle transducer; and measures a specific resistance or conductance generated by an electrochemical or bio-affinity reaction at the microneedle transducer.

The electrochemical analog front end is preferably dynamically configured to achieve any one of the above-numerated embodiments. Likewise, the inputs are preferably arrayed to operate sequentially or in parallel to expand the sensing capabilities of the system.

The wireless transceiver wirelessly relays electrical signals generated by the electrochemical analog front end to a mobile or wearable device using any one of a number of standardized wireless transmission protocols (Bluetooth, WiFi, NFC, RFID, Zigbee, Ant+). Optionally, the electrical signal generated by the analog front end can be amplified, filtered, and/or undergo analog-to-digital-conversion and further signal processing prior to being relayed by the wireless transceiver.

The mobile or wearable device displays sensor readings to the user in an easily-understood format, and performs any additional signal processing necessary.

A method for transducing an electrical signal generated at a microneedle array-based electrochemical biosensor preferably includes the following steps. The application of an electrical probe signal to instigate an electrochemical reaction or measure a change in electrical properties of the biosensor surface. The conversion of a biochemical signal to an electrical signal uses electrochemical techniques such as amperometry, voltammetry, potentiometry, impedimetry, coulometry, or conductimetry, to convert the biochemical signal into an electrical signal whose magnitude or phase is a function of the concentration of the sensed biochemical signal. Biochemical signals from the microneedle array-based electrochemical biosensor preferably comprise a ketone compound, metabolites, electrolytes, hormones, vitamins, minerals, neurotransmitters, and other analytes found in the interstitial fluid, blood, or other physiological media. Concentrations or levels (either relative or absolute) are displayed to the user on an accompanying mobile (phone, tablet) or wearable (smartwatch, fitness band) device.

A method for transducing an electrical signal generated at a microneedle array-based electrochemical biosensor. The method includes pairing a skin-penetrating microneedle array with an electrochemical analog front end to apply a suitable electrical potential or current probe at the microneedle array required to instigate an electrochemical reaction or measure a change in electrical properties at the biosensor surface. The skin-penetrating microneedle array comprises a plurality of protrusions having vertical extent of between 20 and 2000 μm. All or a subset of the plurality of protrusions are functionalized to impart a selective electrochemical biosensing ability. The method also includes measuring a voltage, a current, a frequency, a phase, or a conductivity-based electrical signal generated in response to the electrical probe. The method also includes processing the electrical signal measured by the electrochemical analog front end. The method also includes routing the transduced electrical signal subsequent to the processing operation to a wireless transceiver. The method also includes broadcasting the electrical signal using a standardized wireless transmission format to an external wirelessly-enabled readout device.

The processing includes at least one of amplification, filtering, and analog-to-digital conversion of the signal generated by the electrochemical analog front end.

The electrical potential or current probe preferably embodies steady-state or time-varying properties.

One embodiment is a system for transducing an electrical signal generated at a microneedle array-based electrochemical biosensor. The system comprises a microneedle array electrochemical biosensor, an electrochemical analog front end, a wireless transceiver, and a wearable or mobile device. The microneedle array electrochemical biosensor transduces biochemical signals from an interstitial fluid of a user into a plurality electrical signals. The wireless transceiver wirelessly relays each of the plurality of electrical signals generated by the electrochemical analog front end to the mobile or wearable device using a standardized wireless transmission protocol.

The electrochemical analog front end preferably applies a fixed current or time-varying current to the microneedle array to induce an electrochemical reaction, thereby giving rise to an electrical potential.

The electrochemical analog front end alternatively applies a fixed potential or time-varying potential to the microneedle array to induce an electrochemical reaction, thereby giving rise to an electrical current.

The electrochemical analog front end preferably measures a time-varying open-circuit potential generated by an electrochemical reaction or ionic gradient.

The electrochemical analog front end alternatively measures a frequency-dependent impedance generated by an electrochemical or bio-affinity reaction at the microneedle transducer.

The electrochemical analog front end alternatively measures a specific resistance or conductance generated by an electrochemical or bio-affinity reaction at the microneedle transducer.

The mobile or wearable device preferably displays sensor readings to the user in an easily-understood format.

The microneedle array electrochemical biosensor preferably comprises a plurality of protrusions having vertical extent of between 20 and 2000 p.m. All or a subset of the plurality of protrusions are functionalized to impart a selective electrochemical biosensing ability.

The standardized wireless transmission format is preferably one of Bluetooth, WiFi, NFC, RFID, Zigbee, Ant+, or 4G LTE.

The external wirelessly-enabled readout device is preferably a smartwatch, a fitness tracker, a smartphone, a mobile phone, a tablet computer, or a notebook computer.

The present invention may be utilized with a high-precision and high input impedance analog front end (either a standalone IC or constructed from a series of high input impedance operational amplifiers) cascaded with a high precision integrator and a pair of high input impedance and high (adjustable) gain difference amplifiers to construct a scalable linear-output potentiostat system with sensitivities below 1 nA (100 pA to 700 uA active range). This range can be adjusted via an external gain control. A high-resolution analog-to-digital converter is leveraged to obtain increased signal resolution to the femto- or atto-ampere level.

The high input impedance analog front end, paired with: an adjustable high precision integrator and a pair of mirrored difference amplifier or any variety of such; the use of the mirrored amplifiers and a subtraction algorithm allows the reduction of noise and the removal of fluctuations due to floating or drifting ground issues and external signal ingress; the combined system allows for the detection of extremely low currents without the use of off-board shielding elements (such as a faraday cage); a time average hardware filtering & sampling algorithm also aids in the stabilization of readings by eliminating interfering signal harmonics. A high-resolution analog-to-digital converter can also be leveraged to obtain increased signal resolution to the femto- or atto-ampere level, hence achieving near single-molecule sensitivity.

As shown in FIG. 17, an adjustable bias analog front end/potentiostat 29 is composed of high-input impedance operational amplifiers and a digital to analog converter, or a standalone analog front end (“AFE”) or analog interface integrated circuit package.

An adjustable low noise transimpedance amplifier (“TIA”) converts current flow into a proportional voltage signal, which is adjustable through manual component selection or electronically controlled, and is configured for linear gain (TIA) or integration (integrator) via the implementation of a bypass capacitor.

A mirrored (inverted input) high input impedance and high (adjustable) gain difference amplifier is adjustable through physical resistors (a series of components—multiplexers, relays, and other signal paths—or a physically adjustable potentiometer) or electronically controlled resistors (digital potentiometers), and is configured as a base difference amplifier or any variety of such, including an instrumentation amplifier. Depending on the voltage polarity of the AFE and TIA combination, one amplifier will represent the signal and the second will represent any present ground interference or biases.

Signal filtering eliminates signal ripple due to electro-magnetic interference (“EMI”) following difference amplifier, and is implemented with active or passive low pass, high pass, band pass, or any combination thereof.

A high-resolution analog-to-digital converter is leveraged to convert the filtered analog signal to a precisely quantified value and used to obtain an increased signal resolution to the femto- or atto-ampere level.

A sampling algorithm involves time-average sampling plus offset. The opposing difference amplifier is used to subtract any ground offsets caused by EMI, removing the requirement for external shielding cages or true ground connections.

FIG. 18 is a circuit diagram of a multi-component potentiostat 330 with an electrochemical cell 31.

The method steps of the potentiostat operation are as follows:

The Analog Front End/Potentiostat Operation. The potentiostat/AFE unit consists of either two (FIG. 17) or three (FIG. 18) precision instrumentation operational amplifiers (A1/OA1, OA2, and TIA/OA3) configured in the following arrangement: control amplifier A1/OA1 amplifies the differential voltage (V_{X} in FIG. 9) measured between a variable (programmable) bias and ground (with gain A) and supplies current through the counter electrode (CE). Upon sensing a voltage generated at the reference electrode (RE), A1/OA1 sinks sufficient current in order to maintain its output voltage at the input (V_{RE}) value. In turn, RE is adjusted and the output potential/current of A1/OA2 (a buffer or unity-gain amplifier) is modified accordingly. The control amplifier thus functions as a voltage-controlled current source that delivers sufficient current to maintain the reference electrode at constant potential and arbitrate the electrochemical reaction. In implementing negative feedback, it is imperative that A1/OA2 be able to swing to extreme potentials to allow full voltage compliance required for chemical synthesis. Furthermore, it is crucial that the OA2 possesses very high input impedance in order to draw negligible current; otherwise the reference electrode may deviate from its intended operating potential. In practice, the use of precision amplifiers possessing 20 fA (or lower) of input bias current enables unabated operation to the sub-picoampere level, which is suitable for nearly all electrochemical studies. The TIA/OA3 accepts the current sourced through the working electrode (WE) and outputs a voltage (converted by resistor/capacitor network R_{TIA}/C₅+R₄) proportional to the amount of current passing through electrode WE.

The Analog Front End and Applied Reference/Working Bias. In the system shown in FIGS. 17 and 18, the reference voltage (V_{RE}/RE) is held constant at the inverting and noninverting inputs for operational amplifier A1/OA2, respectively, while the working voltage is changed through a voltage divider, resistor network, or other means, to create an operational bias on the connected sensor. Current passing from CE to WE is directed into the noninverting input of a variable-gain transimpedance amplifier, which converts the current flow into a scaled voltage output (at C2 and/or VOUT/Vo) according to the relation VOUT/Vo=−i_{cell}R_{4/TIA}.

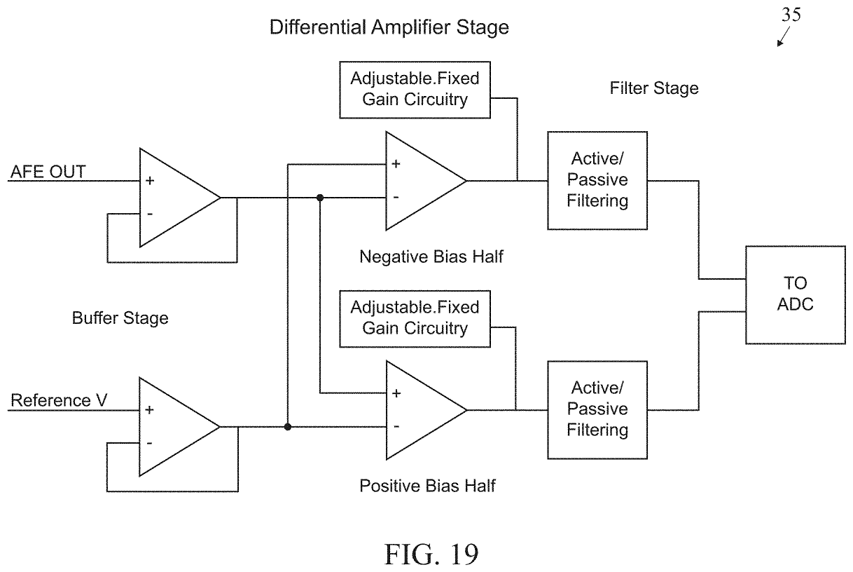

The difference amplifier stage 35 is shown in FIG. 19. The difference amplifiers are configured to accept the applied reference voltage (RE or C1 in the internal IC diagram) and the output from the transimpedance amplifier (with or without a buffer stage). The inputs are juxtaposed among the two amplifiers, namely the reference input is connected to the positive terminal on one of the amplifiers (for negative applied voltages/currents) and on the negative terminal of the other (for positive applied voltages/currents). VOUT is connected to the opposing amplifier input. The unused amplifier (opposing the polarity of the applied current/voltage) will have its inputs driven to zero; it will, however, still possess a ground bias if one is present within the system. The gain of the difference amplifier can be configured either through manufacture or in real time to scale to the amount of voltage/current read in by the AFE.

The Filtering step. The outputs generated from the difference amplifier pair are subsequently subjected to a filtering circuit to remove extraneous noise. Oscillations or random fluctuations in the signal can be present due to a number of reasons, including ground bias, RF interference, mains power oscillation, input impedance mismatch (from the 3 electrode sensor), or from other sources.

The Analog to Digital Converter step. The filtered signals are lastly incident upon an analog to digital converter (“ADC”), either located in an external integrated circuit (“IC”), or co-located within a microcontroller or other IC, and converted into a representative digital signal. Increased sampling resolution may be implemented to gain additional sensitivity and minimize quantization error.

The Collection Algorithm step. To further reduce noise, a time averaged value for both positive and negative bias lines will be collected and computed by a microcontroller/microprocessor over a period of a few seconds (subsequent to digitization by the ADC). The active bias amplifier (applied voltage/current) will have the value of the inactive bias amplifier (ground offset) subtracted in order to remove any present bias in the device. Due to this process, a shielding cage is not required to reach picoampere levels of sensitivity. The inactive bias amplifier, time average data collection, and filtering schemes will provide a stable and scalable output into the microcontroller/processor at all times.

The input of the electrochemical cell or sensor, the analyte, is measured by controlled-potential techniques (amperometry, voltammetry, etc). The output of the sensing system, consisting of a measured voltage and calculated current value (determination of current flowing through working and counter electrodes of electrochemical cell or sensor), corresponds to the concentration of the analyte in the sample.

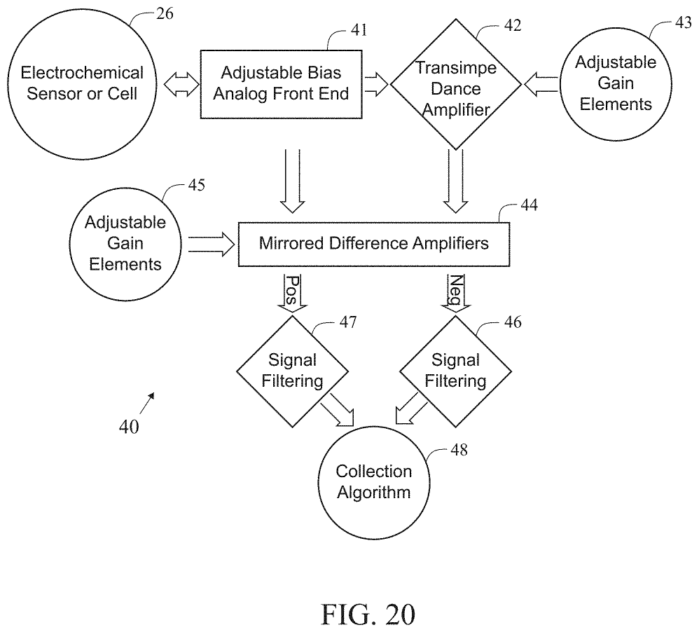

FIG. 20 illustrates a signal flow diagram for detecting a current flowing an electrochemical cell. A current signal from an electrochemical cell 26 is sent to an adjustable bias analog front end 41. The signal is sent to a transimpedance amplifier 42. The signal is sent from both the adjustable bias analog front end 41 and the transimpedance amplifier 42 to mirrored difference amplifiers 44. The outputs generated from the mirrored difference amplifiers 44 are subsequently subjected to filtering circuits 46 and 47 to remove extraneous noise. Oscillations or random fluctuations in the signal can be present due to a number of reasons, including ground bias, RF interference, mains power oscillation, input impedance mismatch (from the 3 electrode sensor), or from other sources. At the collection algorithm 48, to further reduce noise, a time averaged value for both positive and negative bias lines is collected and computed by a microcontroller/microprocessor over a suitable time period, such as a few seconds (subsequent to digitization by the ADC). The active bias amplifier (applied voltage/current) will have the value of the inactive bias amplifier (ground offset) subtracted in order to remove any present bias in the device. Due to this process, a shielding cage is not required to reach picoampere levels of sensitivity. The inactive bias amplifier, time average data collection, and filtering schemes will provide a stable and scalable output into the microcontroller/processor/ADC at all times.

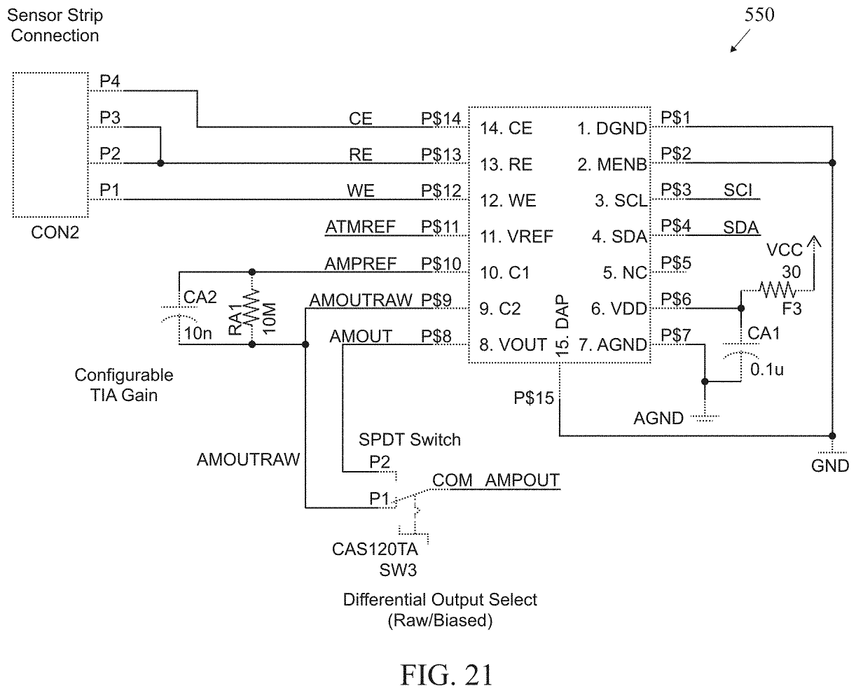

FIG. 21 is a detailed circuit diagram of an integrated analog front end 550 and sensor interface. This is a circuit diagram of an integrated AFE available from a manufacturer that communicates (SCL and SDA lines) with a central microcontroller/microprocessor unit and controls an electrochemical sensor via the CE (counter electrode), WE (working electrode), and RE (reference electrode) lines. The configurable circuit components for the transimpedance amplifier (TIA) are present across 9 and 10 and forms an integrator as configured in the image.

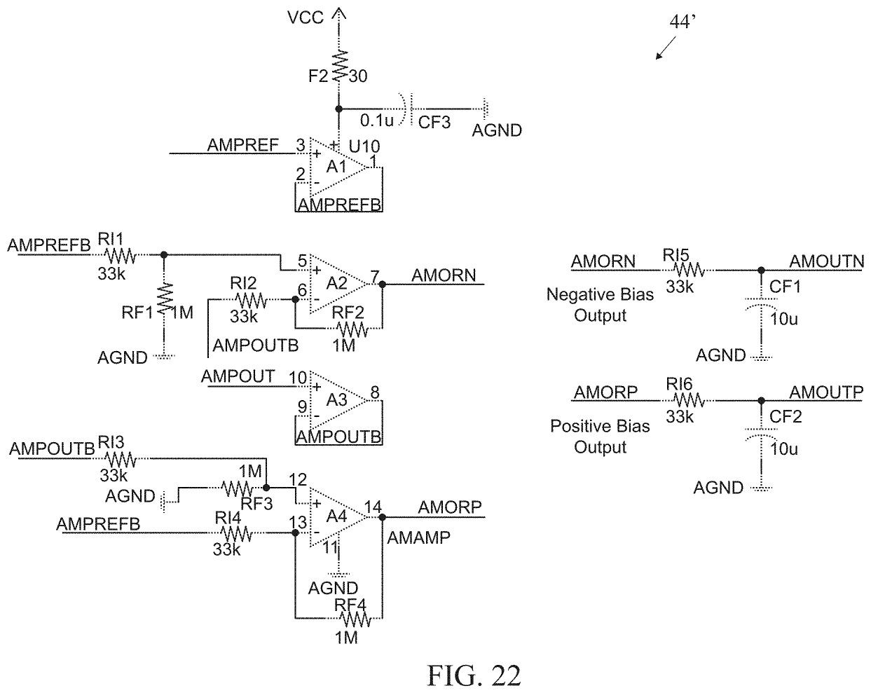

FIG. 22 is a detailed circuit diagram of mirrored difference amplifiers 44′ and filtering. Here, a set of mirrored difference amplifiers is shown utilizing individual operational amplifier components (left side) and a low pass filter on the output(right side). AMORP and AMORN are the positive and negative differential signals, and AMOUTN and AMOUTP are the filtered differential signals. Output gain is controlled by the passive resistors connected to the amplifiers.

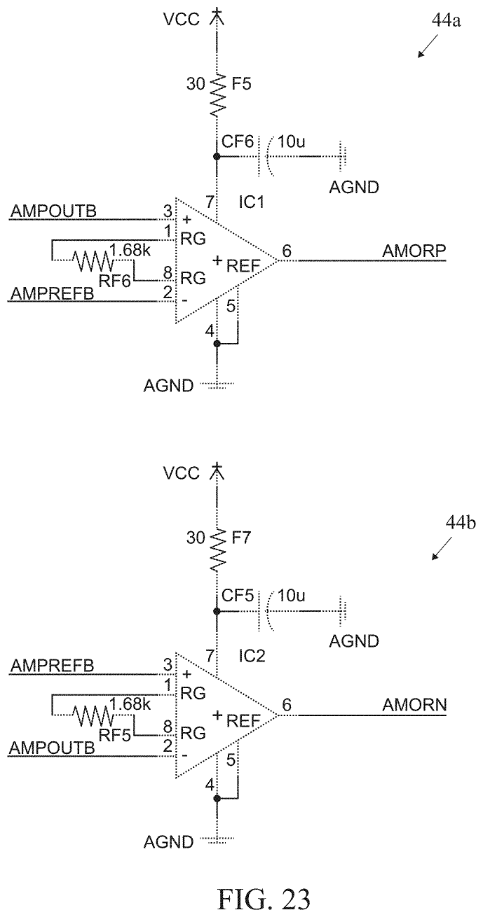

FIG. 23 is a detailed circuit diagram of fixed mirrored instrumentation amplifiers 44a and 44b. Here, a set of mirrored difference amplifiers is shown using a pair of integrated instrumentation amplifiers. Output gain is controlled by a single resistor connected to the RG terminals.

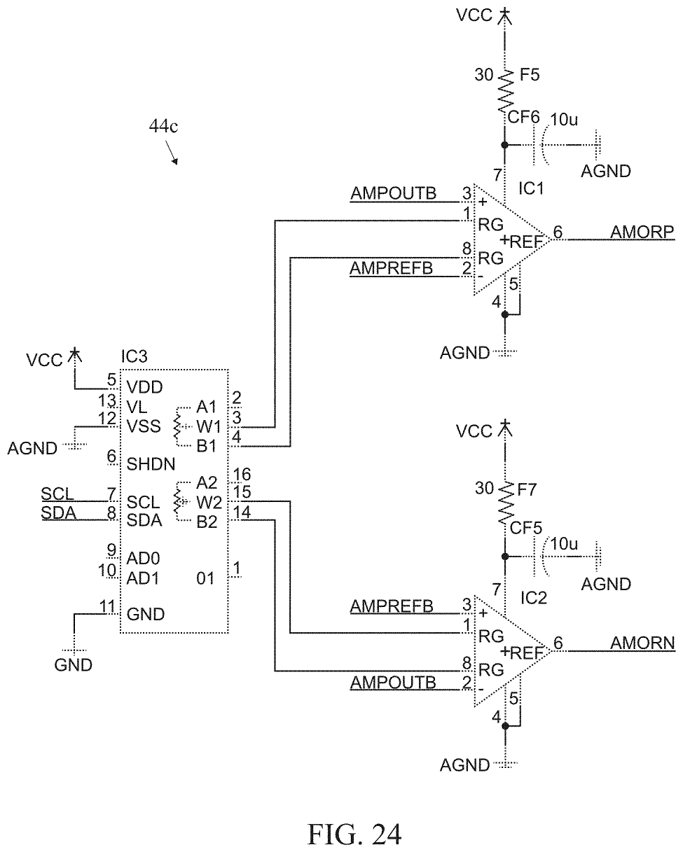

FIG. 24 is a detailed circuit diagram of digital potentiometer-adjustable mirrored instrumentation amplifiers 44c. This is similar to FIG. 23, albeit utilizing a programmable/digitally selectable gain resistor integrated circuit (IC3) rather than passive components.

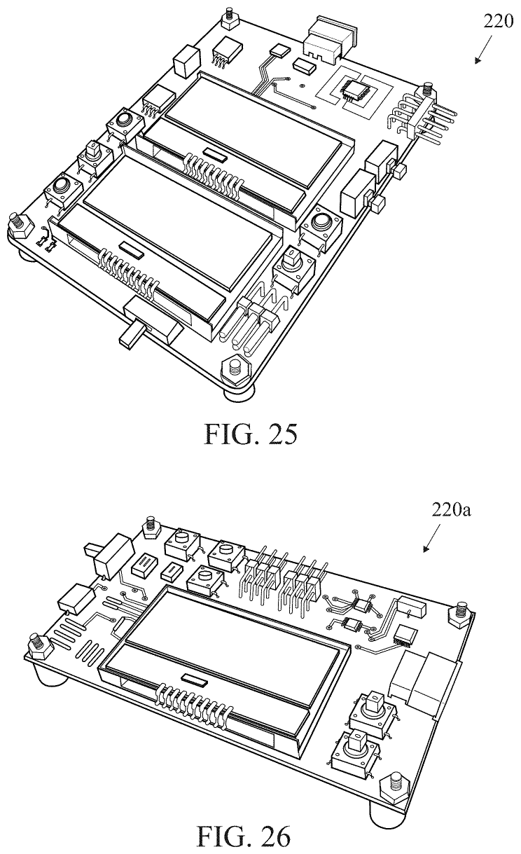

FIG. 25 is an illustration of a handheld analyzer 220 in a large form factor.

FIG. 26 is an illustration of a handheld analyzer 220a in a small form factor.

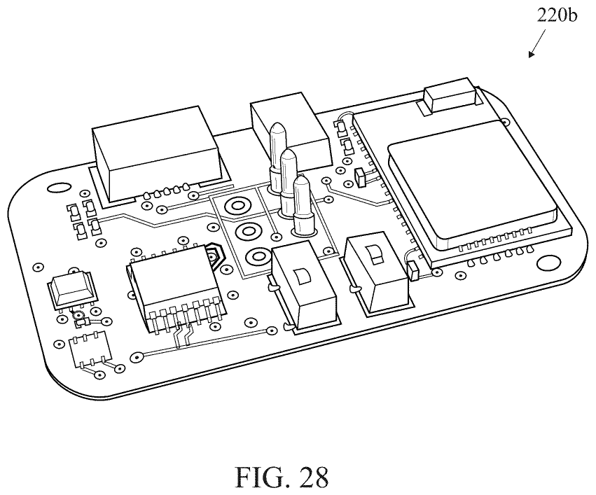

FIG. 28 is an illustration of a handheld analyzer 220b in a small form factor.

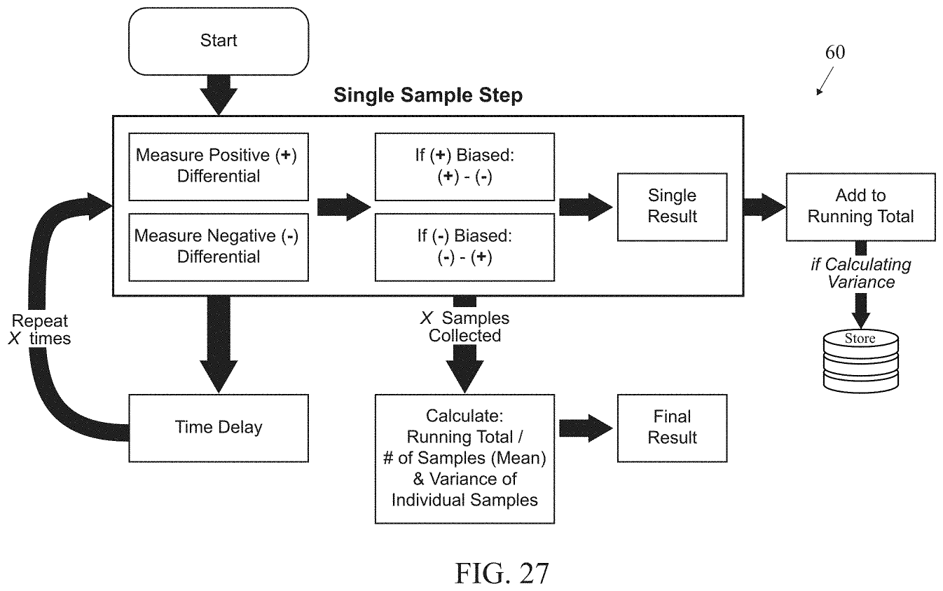

The sampling and measurement algorithm is designed to minimize sources of noise that are not compensated or otherwise removed using the circuit hardware. As shown in the block diagram 60 of FIG. 27, each “sample” involves reading both the positive and negative differential outputs and subtracting one from the other. Multiple samples can be collected and analyzed via statistical operations to yield a measurement. The simplest form is to calculate mean and variance/standard deviation from a set of individual samples. The sampling period has to be selected in a manner that minimizes the possibility of noise from other electrical sources.

The main sources of noise are: floating ground and ground drift; mains power; and high frequency interference.

The floating ground and ground drift are compensated by various means. Floating ground (DC noise) is compensated by the presence of the paired difference amplifiers. Ground drift is compensated by averaging multiple samples. If measuring a positive bias/current, the negative output will be equal to the floating ground. Subtracting the negative output from the positive will remove noise caused by ground drift. The opposite can be performed when measuring a negative bias/current. The subtraction step should be performed at each sample rather than using averages of multiple readings.

Mains Power is also compensated in various ways. Noise arising due to mains power when either connected to an AC power line or induced by proximity to other AC line-powered equipment is compensated by selection of the algorithm sampling period. Sampling should never be performed at the same delay as the period of the line power cycle (16 or 20 ms for 60 Hz and 50 Hz power systems, respectively) or any multiple thereof (i.e. 32 to 40 ms for a multiple of two, etc). If sampling delay is less than the line power cycle (16-20 ms), at least one cycle (at 50-60 Hz) must be captured by multiple samples. For proper statistical analysis, enough samples must be collected to establish an adequate estimate of the standard deviation and mitigate power line harmonics. For a 95% confidence interval for Type 1 (false positive) and Type 2 (false negative) errors, for example, at least 13 samples must be measured. This is application-specific but a minimum of 10 samples is recommended. The maximum sample number is application-dependent (the likelihood of sudden changes due to external factors, such as movement in the case of a body worn sensor).

High frequency interference, noise due to wireless transmission and other high frequency signals, is eliminated fully by hardware filtering, notably low pass filtering.

One embodiment of the device is an enzymatic electrochemical sensor, whereby an enzyme, such as D-β-hydroxybutyrate dehydrogenase, and a cofactor, such as nicotinamide adenine dinucleotide, are immobilized on the sensor surface and, in the presence of a ketone compound, D-β-hydroxybutyrate being on example, will cause a stoichiometric equivalent quantity of cofactor, nicotinamide adenine dinucleotide, to reduce its oxidation state. This reduced form of cofactor (reduced nicotinamide adenine dinucleotide) is subsequently converted to the oxidized form by the application of a bias potential, current, DC signal, AC signal, waveform, optical, or acoustic signal. Following quantization, the magnitude, phase, or other physical quantity of this signal is assessed to determine if it exceeds a pre-defined level, threshold, or rate of change required to generate an alert. Yet another embodiment of the device is a non-enzymatic electrochemical sensor whereby a catalyst, such as a metal or metal oxide, is featured on the sensor or electrode surface and, in the presence of a ketone compound, D-β-hydroxybutyrate being on example, will cause this compound to convert to an electroactive product that may be directly oxidized or reduced at the sensor or electrode surface. With the application of a bias potential, current, DC signal, AC signal, waveform, optical, or acoustic signal at said sensor/electrode, the magnitude, phase, or other physical quantity of this signal is assessed to determine if it exceeds a pre-defined level, threshold, or rate of change required to generate an alert.

Yet another embodiment of the device is a non-enzymatic electrochemical sensor whereby a catalyst, such as a metal or metal oxide, is featured on the sensor or electrode surface and, in the presence of a ketone compound, D-β-hydroxybutyrate being on example, will cause this compound to directly oxidize or reduce. With the application of a bias potential, current, DC signal, AC signal, waveform, optical, or acoustic signal at said sensor/electrode, the magnitude, phase, or other physical quantity of this signal is assessed to determine if it exceeds a pre-defined level, threshold, or rate of change required to generate an alert.

Yet another embodiment of the device is an enzymatic biofuel cell whereby at least one of the anode and cathode contains an enzyme, such as D-β-hydroxybutyrate dehydrogenase, and a cofactor, such as nicotinamide adenine dinucleotide, or a redox mediator, immobilized thereon. In the presence of a ketone compound, D-β-hydroxybutyrate being on example, will cause a stoichiometric equivalent quantity of cofactor, nicotinamide adenine dinucleotide, or mediator to change (reduce or increase) its oxidation state. In conjunction with an oxidation or reduction reaction at the paired electrode of the contingent (anode or cathode) a voltage (electromotive force) will arise between the said anode and cathode due to said oxidation reaction at said anode and said reduction reaction at said cathode. In turn, this electromotive force will cause a current to flow, which is proportional to the magnitude of the redox reaction. Following quantization, the magnitude or other physical quantity of this signal is assessed to determine if it exceeds a pre-defined level, threshold, or rate of change required to generate an alert.

Yet another embodiment of the device is a non-enzymatic fuel cell whereby at least one of the anode and cathode contains an enzyme, such as D-β-hydroxybutyrate dehydrogenase, and a cofactor, such as nicotinamide adenine dinucleotide, or a redox mediator, immobilized thereon. In the presence of a ketone compound, D-β-hydroxybutyrate being on example, will cause a stoichiometric equivalent quantity of cofactor, nicotinamide adenine dinucleotide, or mediator to change (reduce or increase) its oxidation state. In conjunction with an oxidation or reduction reaction at the paired electrode of the contingent (anode or cathode) a voltage (electromotive force) will arise between the said anode and cathode due to said oxidation reaction at said anode and said reduction reaction at said cathode. In turn, this electromotive force will cause a current to flow, which is proportional to the magnitude of the redox reaction. Following quantization, the magnitude or other physical quantity of this signal is assessed to determine if it exceeds a pre-defined level, threshold, or rate of change required to generate an alert.

Yet another embodiment of the device is a colorimetric sensor (optional dye) whereby upon exposure to a ketone compound, D-β-hydroxybutyrate being on example, a color change or color intensity modulation is produced. The magnitude of this change or modulation is assessed to determine if it exceeds a pre-defined level, threshold, or rate of change required to generate an alert.

Yet another embodiment of the device is an optical sensor featuring, optionally, a fluorophore or optically-active intermediary. Upon exposure to a ketone compound, D-β-hydroxybutyrate being on example, a change in absorbance or emission wavelength is produced. The magnitude of this change or is assessed to determine if it exceeds a pre-defined level, threshold, or rate of change required to generate an alert.

An additional embodiment is the generation of a user-prompted alert, alarm, or notification in scenarios wherein the level of a ketone compound or plurality of ketone compounds in the physiological fluid attain or exceed 0.6 mmol/L.

An additional embodiment is the generation of a user-prompted alert, alarm, or notification in scenarios wherein the user's blood (or interstitial) glucose is above some pre-determined value measured by a continuous glucose monitor (e.g. 300 mg/dL) and the level of a ketone compound or plurality of ketone compounds in the physiological fluid are increasing from a baseline of 0.4 mmol/L to a pre-determined level associated with elevated levels of ketones.

An additional embodiment is the generation of a user-prompted alert, alarm, or notification in scenarios wherein the user is engaged in an intravenous, subcutaneous, intramuscular, intradermal, or oral therapy, such as sodium-glucose cotransporter-1/2 inhibitors, and the level of a ketone compound or plurality of ketone compounds in the physiological fluid are increasing from a baseline of 0.4 mmol/L.

An additional embodiment is the generation of a user-prompted alert, alarm, or notification in scenarios wherein the user is undergoing automated insulin delivery and the level of a ketone compound or plurality of ketone compounds in the physiological fluid are increasing gradually.

Yet another embodiment is an alert, alarm, prompt or notification that is visual, auditory, or haptic in nature, or combination thereof.

Yet another embodiment is the generation of an alert, alarm, prompt or notification based on a rate-of-change of the level of a ketone compound or plurality of ketone compounds in the physiological fluid.

Yet another embodiment is the generation of an alert, alarm, prompt or notification based on exceeding a threshold of the level of a ketone compound or plurality of ketone compounds in the physiological fluid.

Yet another embodiment is the generation of an alert, alarm, prompt or notification based on a personalized or generic risk-stratification of a ketone compound or plurality of ketone compounds in the physiological fluid.

Yet another embodiment is the generation of stratified alert levels in scenarios wherein a ketone compound or plurality of ketone compounds in the physiological fluid are within the following ranges: Normal: less than 0.6 mmol/L; Moderate Ketosis/Nutritional Ketosis: between 0.6 and 1.5 mmol/L; DKA Risk: between 1.5 and 3.0 mmol/L; Possible DKA: greater than 3.0 mmol/L.

Yet another embodiment is the presentation of unique colors on a user's display device in scenarios wherein a ketone compound or plurality of ketone compounds in the physiological fluid are within the following ranges: Green: less than 0.6 mmol/L; Yellow: between 0.6 and 1.5 mmol/L; Red: between greater than 1.5 mmol/L.

Yet another embodiment is the presentation of icons, colors, or shapes that are representative of the varying stratifications of risk associated with specified levels of a ketone compound or plurality of ketone compounds.

Yet another embodiment is the generation of unique vibration or haptic patterns that are representative of the varying stratifications of risk associated with specified levels of a ketone compound or plurality of ketone compounds.

Yet another embodiment is the generation of a report delineating the time of day whereby increased levels of a ketone compound or plurality of ketone compounds are measured or increased rates of change for said ketones are measured.

Yet another embodiment is the titration of the automated or user-directed delivery of a therapeutic compound as a consequence of the measurement of the level of a ketone compound or plurality of ketone compounds in the physiological fluid.

Yet another embodiment is the prompting of the user to take measures to prevent the onset of or otherwise treat DKA based on the measured level or rate-of-change of a ketone compound or plurality of ketone compounds in the physiological fluid.

Yet another embodiment is the presentation of a unified glucose-ketone quantitative value to the user, which is derived from a mathematical relation, and indicative of the user's degree of management of their glycemic state.

Yet another embodiment is the presentation of the metabolic system being invoked for the user's energy demands based on the measured level of a ketone compound or plurality of ketone compounds in the physiological fluid. Optionally, this measure may include the level of glucose in the physiological fluid.

Yet another embodiment is the presentation of the user's caloric expenditure based on the measured level of a ketone compound or plurality of ketone compounds in the physiological fluid. Optionally, this measure may include the level of glucose in the physiological fluid.

Yet another embodiment is the incorporation of readings from inertial measurement unit located on a user, such as a smartphone or smartwatch, along with measurements of the level of a ketone compound or plurality of ketone compounds in the physiological fluid in order to advise on physical activity.

Yet another embodiment is the data transmission of measurements of the level of a ketone compound or plurality of ketone compounds in the physiological fluid to a user's support network, healthcare provider, emergency responders, or other relevant stakeholder for assessment.

Yet another embodiment is the data transmission of an alert, alarm, prompt or notification based on the absolute level, rate-of-change of said level, or if a threshold level is exceeded of a ketone compound or plurality of ketone compounds in the physiological fluid to a user's support network, healthcare provider, emergency responders, or other relevant stakeholder for assessment.

Yet another embodiment is the data transmission of the absolute level, rate-of-change of said level, or if a threshold level is exceeded of a ketone compound or plurality of ketone compounds in the physiological fluid to a user's connected mobile (i.e. smartphone) or wearable (i.e. smartwatch) device.

Yet another embodiment is the data transmission of an alert, alarm, prompt or notification based on the absolute level, rate-of-change of said level, or if a threshold level is exceeded of ketone compounds in the physiological fluid to a user's connected mobile (i.e. smartphone) or wearable (i.e. smartwatch) device.

Yet another embodiment is the ability of the user to enable and disable continuous data display, alerts, and/or notifications.

Yet another embodiment is the presentation of a time-series trace to the user on a display device both glucose level in the physiological fluid and the level of a ketone compound or plurality of ketone compounds in the physiological fluid.

Yet another embodiment is dynamic or otherwise adaptive notifications wherein users identified as low risk (i.e. normal CGM readings, no SGLT-2 therapy) are only presented with an alert, alarm, prompt or notification based when a threshold level is exceeded of a ketone compound or plurality of ketone compounds in the physiological fluid.

Yet another embodiment is the generation of audible or haptic alerts and notifications that are each unique to the glucose level in the physiological fluid and the level of a ketone compound or plurality of ketone compounds in the physiological fluid.

Yet another embodiment is the presentation of a quasi-continuous measure of a ketone compound or plurality of ketone compounds in the physiological fluid.

Yet another embodiment is the generation of risk-stratified alarms, alerts, or notifications if the user is: (1) using an insulin pump and therefore presents a greater risk hypoinsulemia due to an insulin pump or infusion set malfunction; (2) taking a therapeutic, such as an SGLT-2 inhibitor, and presenting with a higher basal level of circulating ketone bodies even when in euglycemia; (3) administering insulin and hence likely to only be covered by a basal insulin during at certain times; (4) distracted and thereby neglects to administer insulin; or (5) subject to a diurnal pattern such that there are defined periods of carbohydrate restriction or ingestion.

Yet another embodiment is the generation of an alarm when the measured level of a ketone compound or plurality of ketone compounds in the physiological fluid exceeds 0.6 mmol/L.

Yet another embodiment is the generation of an alarm, alert, or notification when the measured level of a ketone compound or plurality of ketone compounds in the physiological fluid exceeds 0.6 mmol/L.

Yet another embodiment is the generation of an alarm, alert, or notification when the measured level of glucose in the physiological fluid exceeds 240 mg/dL and the level of a ketone compound or plurality of ketone compounds in the physiological fluid increases from a baseline level of 0.4 mmol/L.

Yet another embodiment is the generation of an alarm, alert, or notification when the user is on orally administered therapeutic agents (i.e. SGLT-2 inhibitors) and the level of a ketone compound or plurality of ketone compounds in the physiological fluid increases from a baseline level of 0.4 mmol/L irrespective of measured levels of glucose.

Yet another embodiment is the generation of an alarm, alert, or notification when the user is on insulin infusion therapy and the level of a ketone compound or plurality of ketone compounds in the physiological fluid increases from a baseline level of 0.4 mmol/L irrespective of measured levels of glucose.

Yet another embodiment is the implementation of an electrochemical sensor in a microneedle array configured to perform ketone quantification in the viable epidermis or dermis.

Yet another embodiment is the implementation of two electrochemical sensors in a microneedle array configured to perform glucose and ketone quantification in the viable epidermis or dermis.

Yet another embodiment is the implementation of a plurality of electrochemical sensors in a microneedle array configured to perform ketone and analyte quantification in the viable epidermis or dermis.

Yet another embodiment is the implementation of an electrochemical ketone sensor on one microneedle array and the implementation of an electrochemical glucose sensor on a second distinct microneedle array for the purpose of ketone and glucose quantification, respectively, in the viable epidermis or dermis.

Yet another embodiment is the implementation of an electrochemical sensor in a subcutaneous sensor configured to perform ketone quantification in the adipose tissue of the subcutis.

Yet another embodiment is the implementation of two electrochemical sensors in a subcutaneous sensor configured to perform glucose and ketone quantification in the adipose tissue of the subcutis.

Yet another embodiment is the implementation of a plurality of electrochemical sensors in a subcutaneous sensor configured to perform ketone and analyte quantification in the adipose tissue of the subcutis.

McCanna et al., U.S. Pat. No. 9,933,387, for a Miniaturized Sub-Nanoampere Sensitivity Low-Noise Potentiostat System is hereby incorporated by reference in its entirety.

Windmiller et al., U.S. patent application Ser. No. 14/955,850, filed on Dec. 1, 2015, for a Method And Apparatus For Determining Body Fluid Loss is hereby incorporated by reference in its entirety.

Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.

Wang et al., U.S. Patent Publication Number 20140336487 for a Microneedle Arrays For Biosensing And Drug Delivery is hereby incorporated by reference in its entirety.

Windmiller, U.S. Pat. No. 10,092,207 for a Tissue Penetrating Electrochemical Sensor Featuring A Co Electrodeposited Thin Film Comprised Of A Polymer And Bio-Recognition Element is hereby incorporated by reference in its entirety.

Windmiller, et al., U.S. patent application Ser. No 15/913,709, filed on Mar. 6, 2018, for Methods For Achieving An Isolated Electrical Interface Between An Anterior Surface Of A Microneedle Structure And A Posterior Surface Of A Support Structure is hereby incorporated by reference in its entirety.

PCT Application Number PCT/US17/55314 for an Electro Deposited Conducting Polymers For The Realization Of Solid-State Reference Electrodes For Use In Intracutaneous And Subcutaneous Analyte-selective Sensors is hereby incorporated by reference in its entirety.

Windmiller et al., U.S. patent application Ser. No. 15/961,793, filed on Apr. 24, 2018, for Heterogeneous Integration Of Silicon-Fabricated Solid Microneedle Sensors And CMOS Circuitry is hereby incorporated by reference in its entirety.

Windmiller et al., U.S. patent application Ser. No. 16/051,398, filed on Jul. 13, 2018, for Method And System For Confirmation Of Microneedle-Based Analyte-Selective Sensor Insertion Into Viable Tissue Via Electrical Interrogation is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A body-worn sensor configured to measure the levels of a ketone compound circulating in a physiological fluid of a wearer and capable of generating an alert to said wearer if the level of said circulating ketone compound exceeds a pre-defined level or rate of change.

2. The device of claim 1, wherein said sensor includes at least one of an electrochemical sensor, an optical sensor, a galvanic sensor, a voltammetric sensor, an amperometric sensor, a potentiometric sensor, an impedimetric sensor, a resistive sensor, a capacitive sensor, an ultrasonic sensor, a radio-frequency sensor, or a microwave sensor.

3. The device of claim 1, wherein said ketone compound includes at least one of acetone, acetoacetic acid, or β-droxybutyric acid.

4. The device of claim 1, wherein said physiological fluid includes at least one of blood, serum, plasma, interstitial fluid, dermal interstitial fluid, extracellular fluid, intracellular fluid, or cerebrospinal fluid.

5. The device of claim 1, wherein said sensor is also configured to measure glucose circulating in a physiological fluid of the wearer.

6. The device of claim 1, wherein said alert is at least one of a visual notification, audible notification, haptic notification, or a textual notification.

7. The device of claim 1, wherein said pre-defined level includes a threshold value, a value indicative of clinical ketosis, a value indicative of clinical ketoacidosis, a value indicative of metabolic ketosis, a value indicative of diabetic ketoacidosis, a value indicative of metabolic ketoacidosis, or a value indicative of nutritional ketosis.

8. The device of claim 1, wherein said rate of change includes a derivative value or a slope value.

9. A body-worn sensor configured to measure the levels of a ketone compound circulating in a physiological fluid of a wearer and capable of displaying to said wearer a continuous or quasi-continuous reading of said ketone compound circulating in said physiological fluid.

10. The device of claim 9, wherein said sensor includes at least one of an electrochemical sensor, an optical sensor, a galvanic sensor, a voltaminetric sensor, an amperometric sensor, a potentiometric sensor, an impedimetric sensor, a resistive sensor, a capacitive sensor, an ultrasonic sensor, a radio-frequency sensor, or a microwave sensor.

11. The device of claim 9, wherein said ketone compound in ides at east one of acetone, acetoacetic acid, and β-hydroxybutyric acid.

12. The device of claim 9, wherein said physiological fluid includes at least one of blood, serum, plasma, interstitial fluid, dermal interstitial fluid, extracellular intracellular fluid, or cerebrospinal fluid.

13. The device of claim 9, wherein said sensor is also configured to measure glucose circulating in a physiological fluid of the wearer.

14. The device of claim 9, wherein said reading is at least one of a numerical value, a textual notification, and a symbolic notification.

15. A method of generating an alert to a wearer of a body-worn sensor, said alert indicative of a metabolic state of an elevated ketone compound.

16. The method of claim 15, wherein said alert is at least one of a visual notification, audible notification, haptic notification, or a textual notification.

17. The method of claim 15, wherein said wearable device includes at least one of a transdermal sensor, transcutaneous sensor, an intradermal sensor, an intracutaneous sensor, a subdermal sensor, or a subcutaneous sensor.

18. The method of claim 15, wherein said sensor operates by optical, electrical, or electrochemical means.

19. The method of claim 15, wherein said metabolic state includes a state of baseline health, a state of normal health, a state of ketosis, or a state of ketoacidosis.

20. The method of claim 15, wherein said ketone compound includes at least one of acetone, acetoacetic acid, or β-hydroxybutyric acid.

21. A method for determining the rising levels of circulating ketone bodies in physiological fluids, the method comprising:
measuring a concentration of a ketone compound circulating in a physiological fluid of a wearer of a body-worn sensor device comprising one of an electrochemical sensor, an optical sensor, a galvanic sensor, a voltammetric sensor, an amperometric sensor, a potentiometric sensor, an impedimetric sensor, a resistive sensor, a capacitive sensor, an ultrasonic sensor, a radio-frequency sensor, or a microwave sensor;
storing the measurement in a memory of the body-worn device;
determining if the concentration level exceeds a pre-defined level, threshold, or rate of change from a previous measurement; and
generating an alert if the concentration level exceeds a pre-defined level, threshold, or rate of change from a previous measurement.

22. The method according to claim 21 wherein the physiological fluid includes at least one of blood, serum, plasma, interstitial fluid, dermal interstitial fluid, extracellular fluid, intracellular fluid, or cerebrospinal fluid

23. The method according to claim 21 wherein the body-worn sensor device further comprises a processor.

24. The method according to claim 23 wherein the processor of the body-worn sensor device is also configured to measure glucose circulating in a physiological fluid of the wearer.

25. The method according to claim 21 wherein the body-worn sensor device is configured to generate the alert.

26. The method according to claim 25 wherein the alert is at least one of a visual notification, audible notification, haptic notification, or a textual notification.

27. The method according to claim 21 wherein the body-worn sensor device further comprises a wireless transceiver.

28. The method according to claim 21 wherein the body-worn sensor device further comprises a graphical user display.

29. The method according to claim 21 the body-worn sensor device further comprises at least one of a transdermal sensor, transcutaneous sensor, an intradermal sensor, an intracutaneous sensor, a subdermal sensor, or a subcutaneous sensor.

30. The method according to claim 25 wherein the alert is indicative of a metabolic state of an elevated ketone compound.

31. A method of generating an alarm to a person with diabetes informing the person of an increased risk of increased and/or elevated ketone levels and alerting the person to the need for treatment to prevent progression to diabetic ketoacidosis, the method comprising:
utilizing a sensor for continuous and autonomous detection of ketone levels in a physiological fluid of the person, wherein the sensor is integrated into a body-worn element having a primary purpose of the continuous measurement of another analyte; and
generating an alert if the ketone level is above a pre-determined threshold.

32. The method according to claim 31 wherein a simultaneous measurement of ketones and glucose is from the body-worn element in a subcutaneous adipose tissue sensor.

33. The method according to claim 31 wherein a simultaneous measurement of ketones and glucose is from the body-worn element in a intradermal sensor.

34. The method according to claim 31 wherein each of a plurality of glucose readings is continuously updated to a display to the person, and each of a plurality of ketone readings is continuously monitored but displayed to the user only on demand or when the a value exceeds a pre-determined threshold.

35. The method according to claim 31 wherein the alert is an audible alert or a tactile alert.

36. The method according to claim 31 wherein the alert is transmitted to a designated caregiver using a remote monitoring including a plurality of cloud-based notifications.
